(19) United States
(12) Patent Application Publication
(10) Pub. No.: US 2002/0188323 A1
(43) Pub. Date: Dec. 12, 2002

(54) Methods, systems and devices for in vivo electrochemical production of therapeutic agents

(75) Inventors: Avi Penner, Tel Aviv (IL); Eyal Doron, Kiryat Yam (IL); Eliezer Gileadi, Harzllya Pituach (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Remon Medical Technologies Ltd.

(21) Appl. No.: 09/847,341

(22) Filed: May 3, 2001

Related U.S. Application Data
(60) Provisional application No. 60/276,515, filed on Mar. 19, 2001.

(51) Int. Cl. A61N 1/00

(52) U.S. Cl. 607/2

(57) ABSTRACT

A medical implant for producing a therapeutic agent in a body. The medical implant comprises a pair of electrodes and a mechanism for creating an electrical potential between pairs of the electrodes. The electrical potential serves for electrochemically converting at least one substance present in the body, directly or indirectly, into the therapeutic agent.

This application claims the benefit of priority from U.S. Provisional Patent Application No. 60/276,515, filed Mar. 19, 2001.

### FIELD AND BACKGROUND OF THE INVENTION

The present invention relates to methods, systems and devices for in vivo electrochemical production of therapeutic agents and, more particularly, to methods, systems and devices for in vivo electrochemical production of therapeutic agents from body constituents and/or systemically or locally administered constituents.

Electrochemistry is the branch of chemistry that deals with the chemical changes produced by electricity and the production of electricity by chemical changes. Many spontaneously occurring reactions liberate electrical energy, and some of these reactions are used in batteries and fuel cells to produce electric power. Conversely, electric current can be utilized to bring about many chemical reactions that do not occur spontaneously.

While electrochemistry is extensively applied in many technological fields, its application in vivo is limited to fewer reports and applications.

Eelectrochemical treatment of tumors is referred to in the medical literature as ECT.

In an ECT procedure, electrodes are implanted at spaced positions in or around the malignant tumor to be treated. Applied across these electrodes is a low dc voltage usually having a magnitude of less than 10 volts, causing a current to flow between the electrodes through the tumor. Due to an electrochemical reaction, reaction products are yielded which include cytotoxic agents that act to destroy the tumor.

In the ECT technique disclosed by Li et al., in Bioelectromagnetic 18:2-7 (1997), in the article “Effects of Direct Current on Dog Liver: Possible Mechanisms For Tumor Electrochemical Treatment” two platinum anode and cathode electrodes were inserted in a dog's liver with a 3 cm separation therebetween. Applied across these electrodes was a dc voltage of 8.5 volts, giving rise to an average current through the liver of 30 mA. This was continued for 69 minutes, with a total charge of 124 coulombs.

The concentration of selected ions near the anode and cathode were measured. The concentration of Na⁺ and K⁺ ions were found to be higher around the cathode, whereas the concentration of Cl⁻ ions was higher around the anode. Water content and pH were determined near the anode and cathode, the pH values being 2.1 near the anode and 12.9 near the cathode. The released gases were identified as chlorine at the anode and hydrogen at the cathode. The series of electrochemical reactions which took place during ECT resulted in the rapid and complete destruction of both normal and tumor cells in the liver.

Another example of ECT appears in the article “Electrochemical Treatment of Lung Cancer” by Xin et al. in Bioelectromagnetics 18:8-13 (1997). In this ECT procedure platinum electrodes were inserted transcutaneously into the tumor, the voltage applied thereto being in the 6-8 volt range, the current being in the 40 to 100 mA range, and the electric charge, 100 coulombs per cm of tumor diameter.

According to this article, the clinical results indicate that ECT provides a simple, safe and effective way of treating lung cancers that are surgically inoperable and are not responsive to chemotherapy or radiotherapy.

Also disclosing ECT techniques are Chou et al., Bioelectromagnetics 18:14-24 (1997); Yen et al., Bioelectromagnetics 20:34-41 (1999); Turler at al., Bioelectromagnetics 21:395-401 (2000); Ren at al., Bioelectromagnetics 22:205-211 (2001); U.S. Pat. No. 5,360,440 to to Andersen and U.S. Pat. No. 6,021,347 to Herbst et al.

Electrochemical reactions as a function of pH and electrode potential can be predicted by means of a Pourbaix diagram, as disclosed in the Atlas of Electrochemical Equilibria in Aqueous Solutions—Pergamon Press, 1986—by Pourbaix.

While the U.S. Pat. No. 5,458,627 of Baranowski Jr. et al. does not relate to ECT but to the electrochemically controlled stimulation of osteogenesis, it is nevertheless of prior art interest, for it discloses that reaction products produced by an electrochemical reaction includes not only hydrogen and oxygen, but also hydrogen peroxide.

In the text Methods in Cell Biology, Vol. 46—Cell Death—published by Academic Press, it is noted (page 163), that hydrogen peroxide has been reported to be an inducer of cell death in various cell systems. This type of cell death is attributed to the direct cytotoxicity of H₂O₂ and other oxidant lo species generated from H₂O₂.

The above described ECT technologies are limited in several aspects. First, they all pertain to the treatment of solid tumor masses, yet other applications are not envisaged. Second, they all fail to teach implantable electrochemical devices which are controlled and/or powered via telemetry.

U.S. Pat. Nos. 5,797,898 and 6,123,861 to Santini Jr. et al. both describe microchips which comprise a plurality of drug containing capped reservoirs, whereas in one embodiment the release of the drug therefrom is effected by disintegration of the caps via an electrochemical reaction. While Santini Jr. et al. teach an electrochemical in vivo drug release mechanism effected by telemetry, Santini Jr. et al. fails to teach the in vivo electrochemical production of therapeutic agents.

U.S. Pat. No. 6,185,4.55, teaches functional neuromuscular stimulation (FNS) or functional electrical stimulation (FE S) devices, designed also to locally release drugs that inhibit physiological reactions against-the devices.

U.S. Pat. No. 5,938,903 teaches a microelectrode for inserting in vivo, in vitro or in situ into a warm-blooded or cold blooded animal brain or body, or extra-corporeally and measuring intracellular and/or extracellular concentration and/or release and/or reuptake of one or more biogenic chemicals while measuring said chemical in situ, or in vivo or in vitro.U.S. Pat. No. 5,833,715 teaches a pacing lead having a stylet introduced anti-inflammatory drug delivery element advanceable from the distal tip electrode. The element is formed as a moldable biocompatible composite material. The element has a biocompatible matrix material which may be combined with drugs and therapeutic agents to deliver the drugs and agents by co-dissolution or diffusion to the point of either passive or active fixation. The drug delivery element may be rigid and serve to center an active fixation mechanism, preferably a helix, which penetrates the myocardium. U.S. Pat. No. 3,868,578 teaches a method and apparatus for electroanalysis.

U.S. Pat. No. 6,201,991 teaches a method and system for preventing or treating atherosclerosis in which a blood vessel susceptible to or containing atherosclerotic plaque is subjected to a low-frequency electrical impulse at an effective rate and amplitude to prevent or impede the establishment or decrease the size of the plaque in the vessel. The system can be implanted into the body of a patient or applied externally to the skin.

U.S. Pat. No. 5,360,440 teaches an apparatus for the in situ generation of an electrical current in a biological environment characterized by including an electrolytic fluid. The apparatus comprises first and second electrodes of differing electrochemical potentials separated by an insulator. The apparatus is adapted to be implanted in the environment. The presence of the electrolytic fluid and formation of a current path by hyperplastic cells bridging the electrodes enables electrolysis to occur and a direct current to pass through the current path to impede hyperplastic cell growth.

U.S. Pat. No. 6,206,914 teaches an implantable system that includes a carrier and eukaryotic cells, which produce and release a therapeutic agent, and a stimulating element for stimulating the release of the therapeutic agent. The system can also include a sensing element for monitoring a physiological condition and triggering the stimulating element to stimulate the delivery device to release the therapeutic agent. Alternatively, the patient in which the system is implanted can activate the stimulating element to release the therapeutic agent. In one embodiment the carrier is medical electrical electrodes.

Each one of these patents, however, fails to teach in vivo electrochemical production of therapeutic agents.

There is thus a great need for and it would be highly advantageous to have methods, systems and devices for in vivo electrochemical production of therapeutic agents.

### SUMMARY OF THE INVENTION

According to one aspect of the present invention there is provided a method of producing a therapeutic agent in a body, the method comprising implanting a plurality of electrodes in the body and creating an electrical potential between pairs of the electrodes, the electrical potential being for electrochemically converting at least one substance present in the body, directly or indirectly, into the therapeutic agent.

According to another aspect of the present invention there is provided a medical implant for producing a therapeutic agent in a body, the medical implant comprising a pair of electrodes and a mechanism for creating an electrical potential between pairs of the electrodes, the electrical potential being for electrochemically converting at least one substance present in the body, directly or indirectly, into the therapeutic agent.

According to still another aspect of the present invention there is provided a stent comprising a stent body, a pair of electrodes and a mechanism for creating an electrical potential between pairs of the electrodes, the electrical potential being for electrochemically converting at least one substance present in the body, directly or indirectly, into the therapeutic agent. Preferably, the stent body is made, at least in part, of a metal and serves as one of the electrodes.

According to yet another aspect of the present invention there is provided an artificial implantable vessel comprising a vessel body, a pair of electrodes and a mechanism for creating an electrical potential between pairs of the electrodes, the electrical potential being for electrochemically converting at least one substance present in the body, directly or indirectly, into the therapeutic agent. Preferably, the vessel body is made, at least in part, of a metal and serves as one of the electrodes.

According to further features in preferred embodiments of the invention described below, the therapeutic agent is an oxidizing agent.

According to still further features in the described preferred embodiments the oxidizing agent is selected from the group consisting of molecular chloride, perchloric acid, superoxide, ozone, molecular oxygen, singlet oxygen, hydroxyl radical, hypochlorite and hydrogen peroxide.

According to still further features in the described preferred embodiments the therapeutic agent is nitric oxide.

According to another aspect of the present invention there is provided a method of producing an oxidizing agent in a body, the method comprising implanting a plurality of electrodes in the body and creating an electrical potential between pairs of the electrodes, the electrical potential being for electrochemically converting at least one substance present in the body, directly or indirectly, into the oxidizing agent.

According to still another aspect of the present invention there is provided a medical implant for producing an oxidizing agent in a body, the medical implant comprising a pair of electrodes and a mechanism for creating an electrical potential between pairs of the electrodes, the electrical potential being for electrochemically converting at least one substance present in the body, directly or indirectly, into the oxidizing agent.

According to yet another aspect of the present invention there is provided a method of preventing or reducing cell proliferation in a region of a body, the method comprising producing an oxidizing agent in the region of the body by implanting a plurality of electrodes in the body and creating an electrical potential between pairs of the electrodes, the electrical potential being for electrochemically converting at least one substance present in the body, directly or indirectly, into the oxidizing agent in an amount sufficient for reducing cell proliferation in the region of the body.

According to an additional aspect of the present invention there is provided a method of prevention cell proliferation in a body region, the method comprising implanting in the body region or in a blood vessel feeding the body region an implant designed and constructed for the electrochemical production of an oxidizing agent.

According to still an additional aspect of the present invention there is provided a medical implant characterized by preventing cell proliferation in its vicinity via producing an oxidizing agent in a body, the medical implant comprising a pair of electrodes and a mechanism for creating an electrical potential between pairs of the electrodes, the electrical potential being for electrochemically converting at least one substance present in the body, directly or indirectly, into the oxidizing agent.

According to still an additional aspect of the present invention there is provided a method of producing nitric oxide in a body, the method comprising implanting a plurality of electrodes in the body and creating an electrical potential between pairs of the electrodes, the electrical potential being for electrochemically converting at least one substance present in the body, directly or indirectly, into nitric oxide.

According to another aspect of the present invention there is provided a medical implant for producing nitric oxide in a body, the medical implant comprising a pair of electrodes and a mechanism for creating an electrical potential between pairs of the electrodes, the electrical potential being for electrochemically converting at least one substance present in the body, directly or indirectly, into the nitric oxide.

According to still another aspect of the present invention there is provided a method of vasodilating a tissue or organ in a body, the method comprising implanting a plurality of electrodes in the body and creating an electrical potential between pairs of the electrodes, the electrical potential being for electrochemically converting at least one substance present in the body, directly or indirectly, into a vasodilating agent nitric oxide.

According to yet another aspect of the present invention there is provided a method of vasodilating a body region, the method comprising implanting in the body region or in a blood vessel feeding the body region an implant designed and constructed for the electrochemical production of a vasodilating agent nitric oxide.

According to still another aspect of the present invention there is provided a medical implant for vasodilating a tissue or organ in a body, the medical implant comprising a pair of electrodes and a mechanism for creating an electrical potential between pairs of the electrodes, the electrical potential being for electrochemically converting at least one substance present in the body, directly or indirectly, into a vasodilating agent nitric oxide.

According to further features in preferred embodiments of the invention described below, the at least one substance is a normal blood constituent.

According to still further features in the described preferred embodiments the normal blood constituent is selected from the group consisting of chlorine ions, water, hydrogen ions, hydroxide ions, molecular oxygen, nitrite and nitrate ions and L-arginine.

According to still further features in the described preferred embodiments the at least one substance is added to or augmented in the body.

According to still further features in the described preferred embodiments the at least one substance is added to or augmented in the body through a diet.

According to still further features in the described preferred embodiments the at least one substance is added to or augmented in the body through a medical administration.

According to still further features in the described preferred embodiments the at least one substance is nitrite and/or nitrate ions.

According to still further features in the described preferred embodiments creating the electrical potential between the electrodes is effected by a battery in electrical communication with the electrodes.

According to still further features in the described preferred embodiments creating the electrical potential between the electrodes is effected by a telemetric energy transfer from outside the body.

According to still further features in the described preferred embodiments the telemetric energy transfer is selected from the group consisting of radio frequency energy transfer, magnetic energy transfer and acoustic energy transfer.

According to still further features in the described preferred embodiments the mechanism for creating the electrical potential between the electrodes includes a battery in electrical communication with the electrodes.

According to still further features in the described preferred embodiments the mechanism for creating the electrical potential between the electrodes includes an energy transducer for transducing telemetric energy received from outside the body into electric energy.

According to still further features in the described preferred embodiments the telemetric energy is selected from -the group consisting of radio frequency energy, magnetic energy and acoustic energy.

According to still further features in the described preferred embodiments power supply from the battery is controlled via a logic chip communicating therewith or via telemetric energy transfer.

The present invention successfully addresses the shortcomings of the presently known configurations by providing methods, systems and devices for in vivo electrochemical production of therapeutic agents.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several, forms of the invention may be embodied in practice.

In the drawings:

FIG. 1 is a schematic depiction of a system according to the present invention, showing an implant and an optional extracorporeal unit communicating therewith, together with the implant forming a system according to the present invention;

FIG. 2 shows a portion of a stent according to the present invention;

FIGS. 3a-b shows cut view A1 of FIG. 2 and a four-fold magnification thereof, respectively;

FIG. 4 shows a portion of a stent according to another embodiment of the present invention, carrying an elliptic acoustic transducer module in a strut thereof;

FIG. 5 is a schematic depiction of a simple rectifier circuitry employed by an implant, such as a stent, of the present invention;

FIG. 6 is a schematic depiction of an artificial implantable vessel according to the teachings of the present invention;

FIG. 7 is a schematic depiction of an artificial implant for treatment of a tumor mass according to the present invention;

FIG. 8 is a schematic depiction of an implant producing nitric oxide and its placement in the arteries feeding the kidneys with blood;

FIG. 9 is a schematic depiction of an acoustic switch used in context of the present invention; and

FIG. 10 is a schematic depiction of an acoustic switch circuitry used in context of the present invention.

### DESCRIPTION OF THE PREFERRED EMBODIMENTS

The present invention is of methods, systems and devices which can be used for in vivo electrochemical production of therapeutic agents. Specifically, the present invention can be used for in vivo electrochemical production of therapeutic agents from body constituents and/or systemically or locally administered constituents. The present invention is demonstrated herein, in a non limiting fashion, via in vivo electrochemical production of oxidizing agents (hypochlorite and hydrogen peroxide) for preventing unwanted cell proliferation and further via in vivo electrochemical production of the vasodilating agent nitric oxide.

The principles and operation of the methods, systems and devices according to the present invention may be better understood-with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

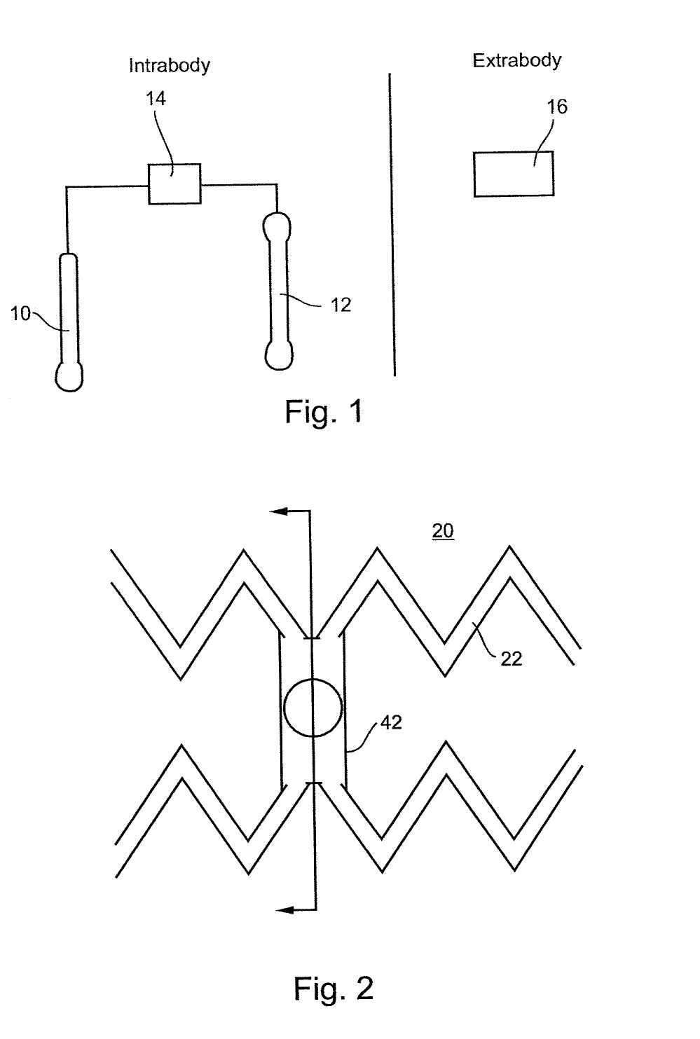

The basic concept underlying the present invention is depicted in FIG. 1, showing a pair of electrodes 10 and 12 and a mechanism 14 for creating an electrical potential between pairs of the electrodes, used for in vivo electrochemical production of therapeutic agents.

According to one aspect of the present invention there is provided a method of producing a therapeutic agent in a body. The method according to this aspect of the invention is effected by implanting a plurality of electrodes in the body and creating an electrical potential between pairs of the electrodes. The electrical potential serves for electrochemically converting at least one substance present in the body, directly or indirectly, into the therapeutic agent.

As used herein throughout, the phrase “a plurality of electrodes” is refers to at least two electrodes, in most embodiments, a pair of electrodes.

As used herein throughout, the phrase “pairs of the electrodes” includes also a single pair of electrodes.

As used herein throughout, the phrase “therapeutic agent” includes agents which, directly or indirectly exert therapeutic benefit.

According to another aspect of the present invention there is provided a medical implant for producing a therapeutic agent in a body. The medical implant comprises a pair of electrodes and a mechanism for creating an electrical potential between pairs of the electrodes. The electrical potential serves for electrochemically converting at least one substance present in the body, directly or indirectly, into the therapeutic agent.

The present invention is exemplified herein, in a non limiting fashion, with respect to the in vivo electrochemical production of the oxidizing agents hypochlorite, hydrogen peroxide, molecular chloride and superoxides, which can be used to prevent unwanted cell proliferation in cases of, for example, cancer, stenosis, restenosis, in-stent stenosis, and in-graft stenosis, and the in vivo electrochemical production of nitric oxide, which is a second messenger, and which have numerous biological functions, among them, vasodilation and wound healing.

As used herein, the phrase “preventing or reducing cell proliferation” is equivalent to “preventing or reducing cell growth” and includes killing cells, inducing tissue necrosis and/or cell apoptosis, and/or inducing cell growth arrest.

Hypochlorite and hydrogen peroxide are strong oxidants and are extensively used as antiseptic agents. Hydrogen peroxide is routinely used as an antiseptic agent in cases of external wounds. Hypochlorite is used as an antiseptic agent during, for example, dental root treatment. Both hypochlorous acid and hydrogen peroxide are well-known disinfectants. Hypochlorite is widely used for treatment of drinking water and as a bactericide. Both hypochlorite and hydrogen peroxide have cytotoxic activity at concentrations of several ppm. Vissers et al. tested the effect of hypochlorite on cells. They noticed a necrosis threshold of 20-40 nmole HClO per 1.2×10⁵ cells, which translates approximately into 2 ppm concentration. Transient growth (proliferation) inhibition occurred at lower concentrations of 5 nmole, or about 0.2-0.3 ppm. See, “Hypochlorous acid causes caspase activation and apoptosis or growth arrest in human endothelial cells”, by Margret C. M. Vissers, Juliet M. Pullar and Mark B. Hampton, Biochem. J. (1999) vol. 344, pp. 443-449. Schraufenstatter et al. discuss the effect of hypochlorite on P388D1 murine macrophage-like tumor cells in mice. They show cell damage at 0.5-1 ppm hypochlorite and cell death from about 4 ppm hypochlorite and up. See, “Mechanisms of Hypochlorite Injury of Target Cells”, by Ingrid U. Schraufenstatter, Ken Browne, Anna Harris, Paul A. Hyslop, Janis H. Jackson, Oswald Quehenberger and Charles G. Cochrane, J. Clin. Invest. vol. 85, February 1990, pp. 554-562.

The discovery and characterization of nitric oxide (NO) as an in vivo ligand has an interesting background. It was demonstrated that the vascular endothelium was not merely the inert lining of blood vessels, but that it was able to influence adjacent smooth muscle in the vessel wall. Removal of the endothelial monolayer from the vessel prevented the production of a relaxing factor, thereby producing contraction. This substance was named endothelial-derived relaxing factor (EDRF) with a half-life of seconds. Its effect on vessel relaxation was blocked in the presence of oxyhaemoglobin and enhanced in the presence of the enzyme superoxide dismutase.

Endogenous vasoactive substances including bradykinin, histamine, serotonin, adenine, nucleotides and shear stress, have all been shown to result in the production of EDRF. In 1987 it was suggested that EDRF was NO because the two compounds had very similar biological properties. Shortly after it was shown that EDRF release from cultured cells required the essential amino acid L-agrinine. Subsequently, it was shown that L-agrinine analogs inhibited nitric oxide release from the vascular endothelium. Nitric oxide has a short half-life and is able to diffuse easily across cell membranes due to its solubility in both water and lipid, enabling it to act as a cell-to-cell messenger. The target for nitric oxide synthesized in a generator cell is soluble guanylate cyclase, an enzyme which catalyses the formation of guanidine cyclic monophosphate (cGMP). Nitric oxide interacts with the heme moiety of guanylate cyclase, activating the enzyme and thereby increasing the intracellular concentration of cGMP. This intracellular second messenger in turn activates protein kinases, which in smooth muscle cells leads to dephosphorylation of the myosin light chains and relaxation. Nitric oxide and cGMP are involved in numerous biological processes, including, but not limited to, control of prostaglandin and prostacyclin production, neurological processes, such as catecholamine release and uptake, modulation of neurosynaptic response, modulation of the immune response, modulation of gut function, modulation of kidney function, and modulation of reproductive and sexual function, such as birth and penile erection. Indeed, the molecule shows influences so many processes that it was chosen as “molecule of the year” by Science magazine in 1992. The association of nitric oxide with diseases related to vasoconstriction is disclosed in U.S. Pat. Nos. 5,132,407; 5,266,594, 5,273,875; 5,281,627 and 5,286,739, its use in treatment of vascular thrombosis and restenosis is described in U.S. Pat. No. 6,063,407. Its use for ovulation control is described in U.S. Pat. No. 5,643,944, and its use for control of wound healing in U.S. Pat. Nos. 6,174,539 and 6,190,704. All of the aforementioned patents are incorporated by reference as if fully set forth herein.

It is demonstrated in the Examples section that follows that therapeutically effective concentrations of oxidizing agents such as hypochlorite and hydrogen peroxide, and of nitric oxide can be readily produced in vivo using electrochemical processes. While the generation of hypochlorite and hydrogen peroxide is from body constituents, normally present in sufficient amounts in the blood (chlorine ions, molecular oxygen and water), the production of nitric oxide may be increased by adding or augmenting at least one substance in the body, through diet or through medical administration, the at least one substance being nitrite and/or nitrate ions, which are present in the blood in limited concentrations.

Creating the electrical potential between the electrodes of an implant according to the present invention can be achieved through several alternatives. In one example, the implant of the invention includes or communicates with a battery being in electrical communication with the electrodes. Miniature body implantable batteries are well known in the art. Such batteries are used, for example, for powering pace-makers and other devices and sensors implanted in the body.

In another example creating the electrical potential between the electrodes of the implant of the invention is effected by telemetric energy transfer from outside the body. As is further detailed herein under, telemetric energy transfer according to the present invention can be effected in any one of a plurality of ways known in the art, including radio frequency energy transfer, magnetic energy transfer and acoustic energy transfer.

Radio frequency energy transfer can be effected, for example, using an antenna coil and a rectifying circuit. Such circuits are well known and in common use in pacemakers and defibrillators, and therefore require no further description herein.

Magnetic energy transfer can be effected, for example, using a magnetic transducer which employs a magnet and a coil as is well known in the art. Examples of magnetic energy transfer are disclosed in, for example, U.S. Pat. Nos. 5,880,661, 6,185,457, 6,167,307, 6,164,284 and 6,162,238, which are incorporated herein by reference.

Acoustic energy transfer can be effected, for example, using an acoustic transducer as described, for example, in U.S. Pat. Nos. 6,140,740 and 6,170,488, which are incorporated herein by reference.

Telemetry can also be used according to the present invention to transmit data pertaining to the implant and/or its effect from within the body outside thereof. Thus, currents, potentials, charges and other parameters can be sensed via suitable sensors included in the implant and/or positioned nearby and the data collected thereby communicated telemetrically outside the body, so as to optimize the dose and the conditions for the electrochemical reaction catalyzed by the implant. The information pertaining to the implant and transmitted outside the body can also include indications for the status of the implant, such as, for example, degradation of the implant electrodes due to corrosion.

If powering the implant is effected with a battery, power supply from the battery to the electrodes is preferably controlled via a preprogrammed logic chip communicating therewith or via telemetric energy transfer to a control energy transducer embedded in the implant, similar to as described above with respect to powering via telemetry. Thus, controlling the device can be effected via acoustic, magnetic or radio frequency telemetry. In a preferred embodiment an acoustic switch is employed. An applicable acoustic switch is described in U.S. patent application Ser. No. 09/690,615, filed Oct. 16, 2000, which is incorporated herein by reference. The design and construction of an acoustic switch are further described in Example 4 of the Examples section that follows.

A logic chip is preferably included in all of the configurations of the implant of the present invention for reasons to be further detailed hereinbelow.

Thus, the implant of the present invention may employ telemetry for accomplishing powering, control and/or communication of data. Different type telemetry can be employed for effecting each of these criteria.

In case telemetry is employed, an extracorporeal unit is provided, designed and constructed for powering, interrogating, controlling and/or receiving data from the implant. The extracorporeal unit is identified in FIG. 1 by reference numeral 16 and together with the implant of the invention forms a system in accordance with the teachings of the invention. To a major extent, the design and construction of the extracorporeal unit depend on its function (e.g., powering, interrogating, controlling and/or receiving data) and the telemetry method employed (e.g., acoustic, magnetic or radio frequency). A single implant of the invention can be designed and constructed to communicate with several extracorporeal units, each serving a different purpose. For example, a home operated powering extracorporeal unit can be employed and operated by the patient carrying the implants, whereas a second extracorporeal unit can be operated by a physician for periodically tuning the activity of the implant in response to powering.

In one embodiment the implant of the present invention forms a part of a stent.

A stent is a tubular element designed and constructed to be placed in a vessel, such as a blood vessel (e.g., a peripheral blood vessel or coronary blood vessel, an artery or vein) or other type vessel, such as the urethra, and provide the vessel with structural support. Stents are typically placed in sections of vessels which were occluded prior to stenting, so as to allow passage of fluid, such as blood, there through.

A major problem associated with stent placement is known as restenosis, or in stent stenosis, which is a proliferative disorder developing several days or weeks post stent placement as a response to the wounding of the vessel in the process of placing the stent. One approach for prevention and/or treatment of restenosis involves slow release of anti-proliferative drugs, such as such as Taxol and/or Rapamycin, from the stent itself (see, for example, U.S. Pat. Nos. 6,171,609 and 6,153,252). This approach has several limitations as follows: (i) drug release starts immediately following stent placement, however, at this point in time the restenosis process has not yet started, and the cytotoxic drug inhibits the natural healing process of the wounded vessel; (ii) the amount of drug is predetermined, no dosage adjustment is possible post stent placement; (iii) not all patients develop restenosis or in stent stenosis, however, in most cases, this cannot be determined in advance; and (iv) the amount of drug loadable on a stent is limited and is exhausted during service.

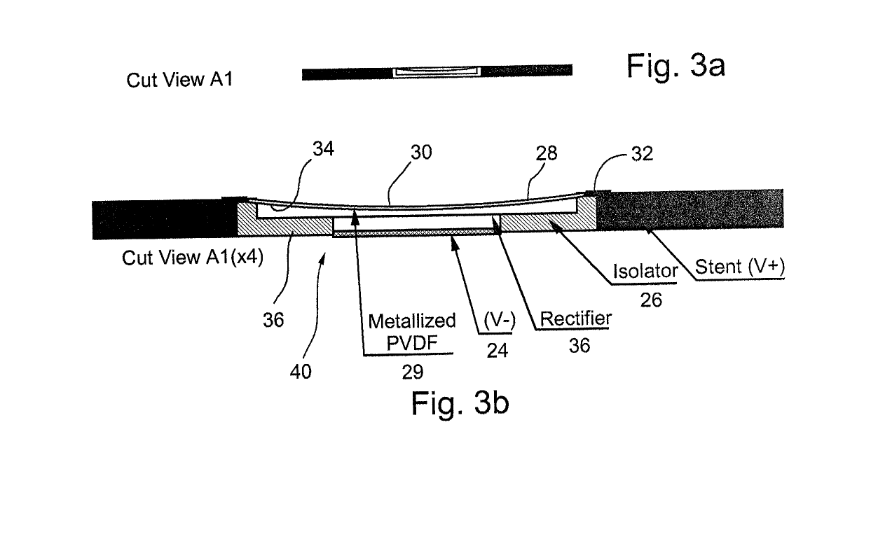

A portion of an electrochemical stent 20 (hereinafter, stent 20) of the present invention is depicted in FIGS. 2 and 3a-b. Stent 20 includes a stent body 22 which is made, at least in part, of, or is coated with, a metal, such as, but not limited to gold, platinum, tantalum or any other electrochemically stable metal or alloy, such that stent body 20 serves as an electrode (V+) for induction of an electrochemical reaction. In the example shown, stent 20 further includes a second electrode (V−) 24 which is electrically isolated from stent body 22 via an isolator 26. Both electrodes 22 and 24 are designed to be in electrical contact with the blood or other body fluids. Stent 20 further includes a mechanism for creating an electrical potential between pairs of the electrodes, which is realized in the specific example shown as an acoustic transducer 28, including a metalized PVDF membrane 29. An external surface 30 of metallized PVDF (positive electrode) membrane 29 is electrically coupled via a conductive coupler 32 to stent body 22. An inner side 34 of PVDF membrane 29 is connected to a rectifier 36 having its output connected to negative electrode 24. Negative electrode 24 is isolated from stent body 22 and an electrical circuit is closed via body fluids, such as blood.

In a presently preferred embodiment of the invention electrode 24, PVDF membrane 29, rectifier 36 and isolator 26 form a self-produceable module 40 receivable within a wall or strut 42 of stent body 22 to form stent 20. Module 40 can be assembled within stent body 22 with negative electrode 24 pointing to the outer surface of stent 20 (i.e., to the vessel wall) or to the inner surface of the stent 20 (i.e., to the blood).

Module 40 can be readily manufactured as small as 0.5 mm in diameter and 0.1 mm in thickness. These dimensions allow its integration within wall or strut 42 of stent body 22. For example, the wall thickness of a coronary stent is 0.1-0.15 mm and that of a peripheral stent is 0.15 mm-0.30 mm.

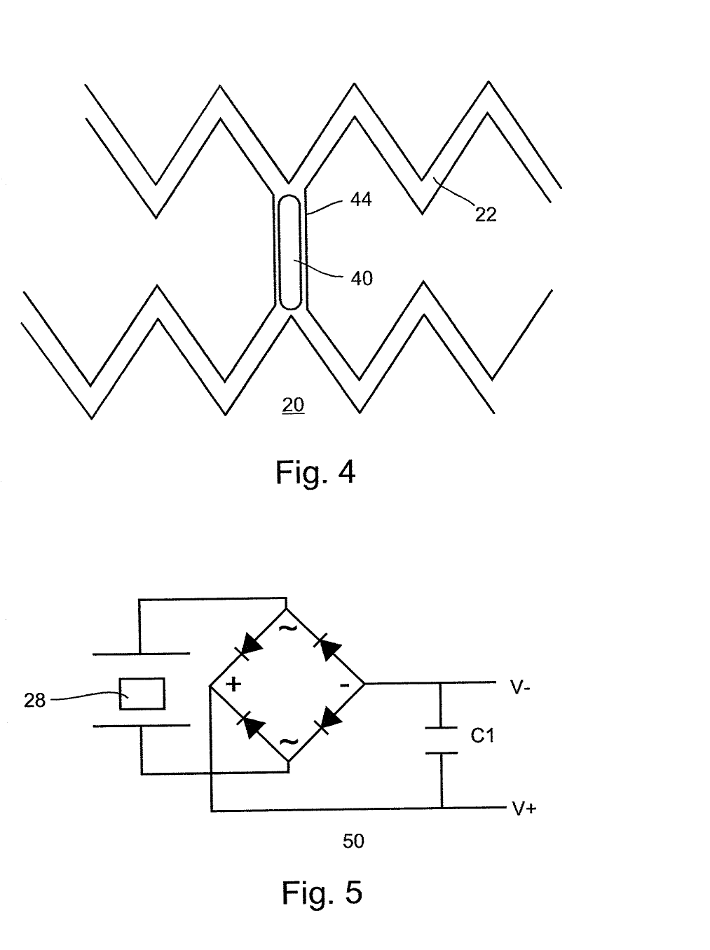

As shown in FIG. 4, module 40 can also be formed in an elliptical shape having a width of about 0.25 mm, a length of about 0.7 and thickness of about 0.1 mm. The elliptical shape allows for better adaptation to a stent's longitudinal strut 44 without loosing acoustic energy conversion efficacy.

Acoustic transducer 28 produces an AC electrical current at a frequency corresponding to the acoustic excitation. An electrochemical reaction, however, requires a DC current (or a low frequency AC current). Rectifier 36 serves to transform the AC current to a DC current. Rectifier 36 can be a simple diode bridge or half bridge or have any other rectifier design.

An example of a rectifier circuit 50 that transforms an AC current produced by module 40 into a DC current is shown in FIG. 5, which is self explanatory. It will be appreciated that capacitor Cl can be eliminated since the capacitance of the double electric layer formed between the electrodes and body fluids is sufficient. The diodes of rectifier 36 can be standard silicon diodes or printed organic diodes (see, for example, U.S. Pat. No. 6,087,196).

In order to protect the electrodes from corrosion one may chose to apply either anodic pulses or smoothly varying anodic current waveforms, in order to maintain an anodic passivating film on the metal surface. For achieving these waveforms a simple rectifier 36 is replaced with a silicon logic chip that includes in addition to the rectifier also the required components and logic.

In case more complex modes of operation are required, such as receiving commands, performing measurements and communicating information outside the body, the simple rectifier is replaced with a silicon chip that includes in addition to the rectifier function also the required components and logic.

During service stent 20 is preferably powered, communicates with and/or controlled via an extracorporeal unit, which, in the example shown, includes one or more acoustic transducers designed and constructed to either power, command and/or control the electrochemical activity of stent 20, and/or to receive data collected via sensors therefrom.

When powered, stent 20 generates oxidizing agents (hypochlorite and/or hydrogen peroxide) in amounts sufficient to prevent cell proliferation, thus, preventing restenosis or in stent stenosis. The amount of oxidizing agents generated depends on the precise configuration and can be controlled by the duration of powering. Similar to systemic administration of anti-proliferative drugs, the stent of the present invention offers the possibility to start anti-proliferative treatment if and when needed, yet in contrast to, and advantageously over, systemic administration, the stent of the present invention offers, like drug slow release stents, the option of local treatment. Advantageously over slow drug release stents, the stent of the present invention offers the possibility to adapt dosages to specific individuals and initiate drug production only when and if so desired. Thus, the physician can choose among complex drug dosages, such as zero-order, pulsatile, bolus or any combination thereof. Anti-proliferative drug treatment using the stent of the invention is not limited by duration of application and/or dosage.

As stated is stated hereinabove, nitric oxide (NO) is a naturally occurring signaling molecule in the body that has many actions, some of them being vasodilatation (relaxation of vessels) and the promotion of endothelial healing. The endothelial cells of the intima naturally constantly produce NO. Nitric oxide triggers a second messenger, cGMP (cyclic guanosine monophosphate) in the intracellular signaling cascade. During PTCA balloon placement the endothelium underlying the inner walls of blood vessels is wounded. Stent placement increases the damage to the endothelium even more. Damage endothelium initiates the inflammation cascade that cause neointimal hyperplasia and blood clots in the artery. Damaged areas that regain a protective endothelial lining are less prone to promote smooth muscle proliferation (neointima hyperplasia). NO is a difficult molecule to deliver using conventional slow release methodologies because it has an extremely short half-life. The stent of the present invention as herein described can be used to produce NO at site of placement, to thereby accelerate the healing proces of the damaged endothelial lining.

Balloon angioplasty per se causes much smaller damage to the vessel as is compared to placement of a stent. As a result the amount of neointima proliferation following balloon angioplasty, as well as thrombus formation are almost not an issue. However, in balloon angioplasty there is a high incident of elastic recoil of the vessel that reduces the vessel open diameter. The present invention offers a solution to this problem, as it is now possible to implant a medical implant having a metallic tubular mesh structure similar to a stent and which is designed to in vivo electrochemically produce NO in order to vasodilatate the vessel and prevent its recoil. Such an implant should not have high radial force or high metallic coverage and, unlike a conventional stent, can be implanted without applying much force on the vessel wall.

Thus, the present invention offers a stent that comprises a stent body, a pair of electrodes and a mechanism for creating an electrical potential is between pairs of the electrodes. The electrical potential serves for electrochemically converting at least one substance present in the body, directly or indirectly, into the therapeutic agent. Preferably, the stent body is made, at least in part, of a metal and serves as one of the electrodes.

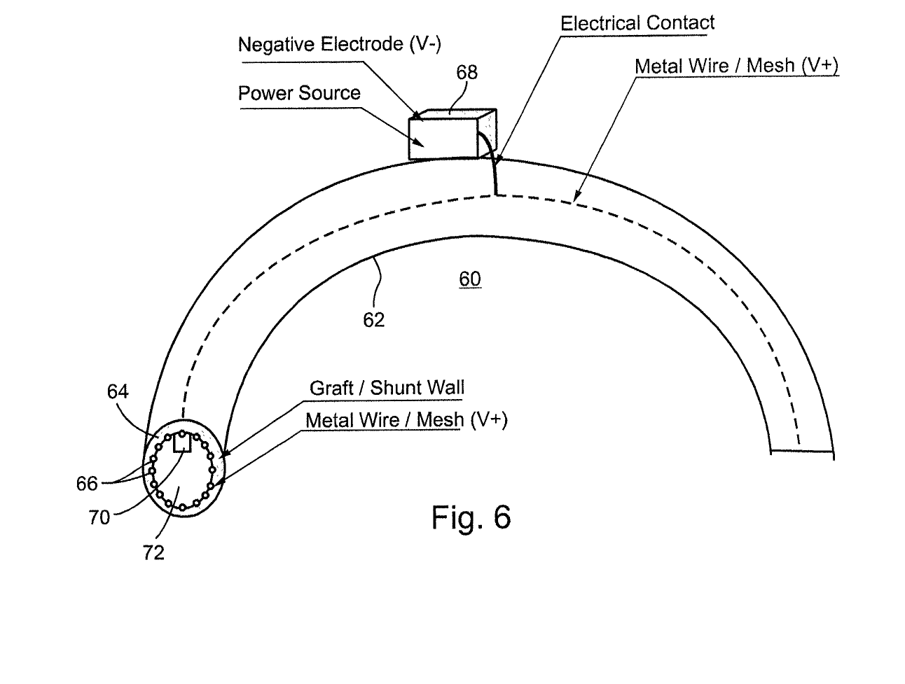

FIG. 6 shows a somewhat different embodiment the present invention. The problem of restenosis is not limited to stents, rather it is also characteristic of artificial implantable vessels, such as blood vessels (known as artificial grafts or by-pass grafts of veins or arteries) and shunts. Thus, an artificial vessel 60 (hereinafter vessel 60) includes a vessel body 62 defining a flexible tube. Body 62 is made of an acceptable material such as ePTFE or Dacron. Embedded within a wall 64 of body 62 is a metal mesh 66, made of, for example, gold, platinum, tantalum or any other electrochemically stable metal or alloy, and which serves as an electrode (V+) in an electrochemical reaction. In the example shown, vessel 60 further includes a second electrode (V−) 68 which is electrically isolated from mesh 66. Both electrodes 66 and 68 are designed to be in electrical contact with body fluids. Vessel 60 further includes a mechanism for creating an electrical potential between electrodes 66 and 68, which mechanism includes, in a preferred embodiment of the invention, an acoustic transducer as is further described hereinabove and in, for example, U.S. Pat. No. 6,140,740. Other telemetry and non-telemetry powering methods, such as magnetic and radio frequency telemetry or a battery are also envisaged. When powered, vessel 60 of the present invention electrochemically generates sufficient amounts of oxidizing agents (hypochlorite and hydrogen peroxide) so as to prevent unwanted cell proliferation therein. A flow sensor 70 can be included in lumen 72 of vessel 60. Sensor 70 preferably includes an energy transducer so as to allow for its powering and/or to communicate data to an extracorporeal unit. Sensor 70 may control via a feedback control loop the electrochemical operation of vessel 60, such that when flow records are indicative of reduced flow due to constriction, the electrochemical production of anti-proliferative antioxidants is increased. In another embodiment sensor 70 measures the electrical impedance between electrodes 66 and 68. This parameter can serve as an indication of cell growth and proliferation.

Thus, the present invention provides an artificial implantable vessel that comprises a vessel body, a pair of electrodes and a mechanism for creating an electrical potential between pairs of the electrodes The electrical potential serves for electrochemically converting at least one substance present in the body, directly or indirectly, into a therapeutic agent. Preferably, the vessel body is made, at least in part, of a metal and serves as one of the electrodes.

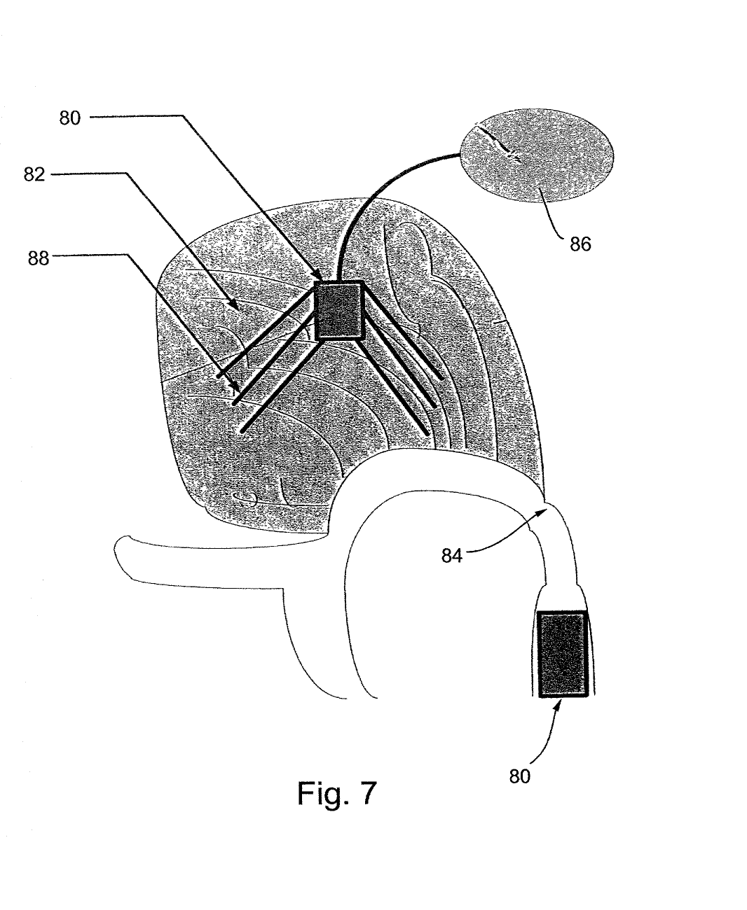

In another embodiment of the present invention, depicted in FIG. 7, an electrochemical implant 80 is used either to produce oxidizing agents within a tumor mass 82 or within a blood vessel 84 feeding tumor mass 82, in which case a stent as described above can be used for placement. Powering is again either via battery 86 or via telemetry as is further described herein. Electrode extensions 88 penetrating mass 82 are preferably employed, so as to expose more cells of mass 82 to the electrochemically produced oxidizing agents.

Thus, the present invention provides a medical implant for producing an oxidizing agent in a body. The medical implant comprises at least a pair of electrodes and a mechanism for creating an electrical potential between pairs of the electrodes, the electrical potential being for electrochemically converting at least one substance present in the body, directly or indirectly, into the oxidizing agent.

In another aspect the present invention provides a method of preventing or reducing cell proliferation in a region of a body. The method comprises producing an oxidizing agent in the region of the body by implanting a plurality of electrodes in the body and creating an electrical potential between pairs of the electrodes. The electrical potential serves for electrochemically converting at least one substance present in the body, directly or indirectly, into the oxidizing agent in an amount sufficient for reducing cell proliferation in the region of the body.

In another aspect the present invention provides a method of prevention cell proliferation in a body region. The method comprises implanting in the body region or in a blood vessel feeding the body region an implant designed and constructed for the electrochemical production of an oxidizing agent.

In yet another aspect the present invention provides a medical implant characterized by preventing cell proliferation in its vicinity via producing an oxidizing agent in a body. The medical implant comprises a pair of electrodes and a mechanism for creating an electrical potential between pairs of the electrodes. The electrical potential serves for electrochemically converting at least one substance present in the body, directly or indirectly, into the oxidizing agent.

As is further discussed hereinabove and exemplified in the Examples section that follows, in vivo electrochemical production of nitric oxide is also within the scope of the present invention. Nitric oxide is a well-known vasodilator. For example, nitric oxide is commonly used for the acute treatment of cardiac ischemia (in this case a nitric oxide donor is used such as nitroglycerin). Electrochemical local production of nitric oxide can also be used for improving kidney functioning of congestive heart failure patients (CHF). One of the most problematic symptoms of CHF patients is the reduction in kidney functions. The body's natural reaction to low cardiac output is vaso-constriction of peripheral blood vessels, including those feeding the kidneys. As a result, the flow through the kidneys decreases, thus reducing the filtration of the blood there through and inducing secondary complications associated with CHF, such as fluid retention.

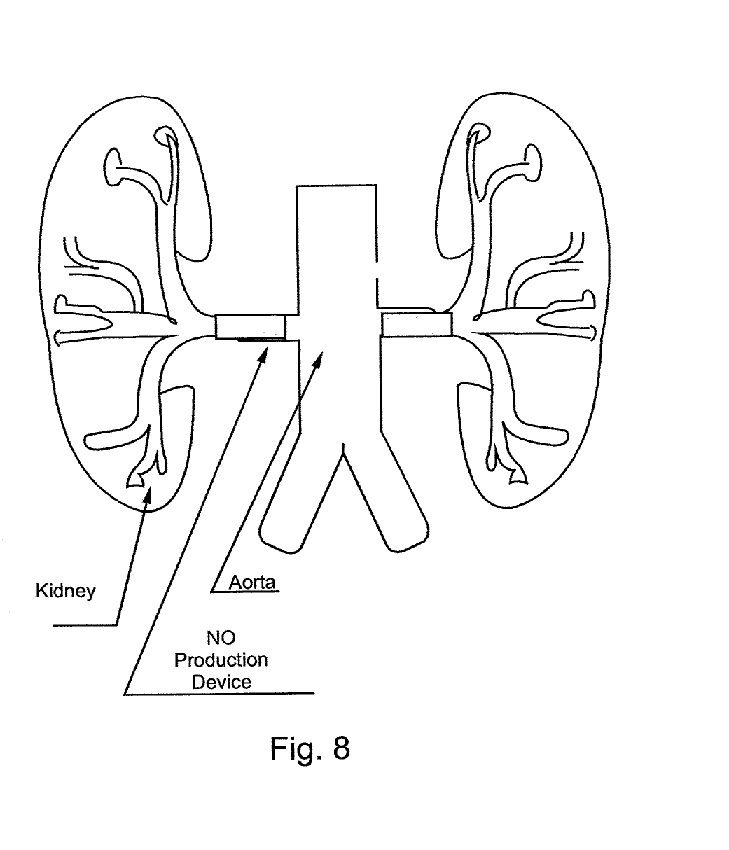

A systemic vasodilation treatment is not applicable since it will reduce the blood pressure of the patient to dangerous levels. However, as shown in FIG. 8, an implant 80 that locally produces a vasodilation agent (nitric oxide) in the kidneys or preferably within the renal artery feeding the kidney will vasodilate only the blood vessels leading to, and within the kidneys, resulting an increase of the flow and filtration of the blood there through. Implant 80 is preferably similar in construction to the above described stent, yet serves to electrochemically convert nitrite and/or nitrate ions into nitric oxide. Although nitrite and/or nitrate ions are normal blood constituents, augmenting their concentration up to the maximal electrode surface concentration of nitric oxide via diet or medical administration (e.g., intravenous injection, tablets) will result in production of nitric oxide which is limited only by the current density over the electrode. Since the maximal electrode surface concentration of nitric oxide is typically in the range of a few ppm, the deleterious effects of the nitrite and/or nitrate ions, if any, will be minimized.

Thus, the present invention provides a method of producing nitric oxide in a body. The method comprises implanting a plurality of electrodes in the body and creating an electrical potential between pairs of the electrodes. The electrical potential serves for electrochemically converting at least one substance present in the body, directly or indirectly, into nitric oxide.

The present invention also provides a medical implant for producing nitric oxide in a body. The medical implant comprises a pair of electrodes and a mechanism for creating an electrical potential between pairs of the electrodes The electrical potential serves for electrochemically converting at least one substance present in the body, directly or indirectly, into the nitric oxide.

The present invention still further provides a method of vasodilating a tissue or organ in a body. The method comprises implanting a plurality of electrodes in the body and creating an electrical potential between pairs of the electrodes. The electrical potential serves for electrochemically converting at least one substance present in the body, directly or indirectly, into a vasodilating agent nitric oxide.

The present invention still further provides a method of vasodilating a body region. The method comprises implanting in the body region or in a blood vessel feeding the body region an implant designed and constructed the electrochemical production of a vasodilating agent nitric oxide. The present invention still further provides a medical implant for vasodilating a tissue or organ in a body. The medical implant comprises a pair of electrodes and a mechanism for creating an electrical potential between pairs of the electrodes. The electrical potential being for electrochemically converting at least one substance present in the body, directly or indirectly, into a vasodilating agent nitric oxide.

The present invention is not limited to the in vivo electrochemical processes described hereinabove and further exemplified hereinbelow in the Examples section, as other electrochemical processes involving body constituents and/or administered constituents may be practiced. Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

Electrochemical Reactions in the Blood and Other Body Fluids—Reactions Involving Species That Naturally Exist in Blood

The average cation and anion composition of human blood is given in Table 1 below.

TABLE 1

| | Concentration mEq. | | |
|---|---|---|---|
| Chemical | Plasma | Interstitial | Intracellular |
| Na⁺ | 142 | 146 | 15 |
| K⁺ | 5 | 5 | 150 |
| Ca⁺⁺ | 5 | 3 | 2 |
| Mg⁺² | 2 | 1 | 27 |
| Total Cations | 154 | 155 | 194 |
| Cl⁻ | 103 | 144 | 1 |
| HCO₃⁻ | 27 | 30 | 10 |
| HPO₄⁻² | 2 | 2 | 100 |
| SO₄⁻² | 1 | 1 | 20 |
| Organic Acids | 5 | 8 | 0 |
| Proteinate | 16 | 0 | 63 |
| Total Anions | 154 | 155 | 194 |

The four cations listed in Table 1 (Na⁺; K⁺; Ca²⁺ and Mg²⁺) are electrochemically inactive in aqueous solutions. Thus, these cations are not discharged when a current or potential is applied.

Of the four anions listed in Table 1 (Cl⁻; HCO³⁻; HPO₄²⁻ and SO₄²⁻) only the chlorine ion is dischargeable at the anode, leading to the production of molecular chloride.

Thus, among all of the inorganic materials that exist in blood (pH 7.4) and other body fluids at significant concentrations, only the following, listed in Table 2 below, are electrochemically active.

TABLE 2

| | | | Reversible E | |
|---|---|---|---|---|
| | Range of | | at pH = 7.4 | |
| Material | concentration | Product | vs. SHE | Comments |
| H₂O | 55 M | H₂ + (OH)⁻ | −0.437 V | Reduction |
| H₂O | 55 M | O_{2 + H}⁺ | +0.792 V | Oxidation |
| H₂O | 55 M | H₂O₂ + H⁺ | +1.339 V | Oxidation |
| Cl⁻ | 0.10-0.15 M | Cl₂ | +1.35 V | Oxidation |
| O₂ | 0.1-0.2 mM | H₂O₂ + (OH⁻) | +0.245 V | Reduction |
| O₂ | 0.1-0.2 mM | H₂O + (OH)⁻ | +0.792 V | Reduction |

The reactions that occur at an anode (the positive electrode) paced in blood are the formation of O₂ and H₂O₂ from water, and the formation of Cl₂ from chloride ions. The latter can interact with water to form HClO. All three reactions also produce H⁺, and may thus cause a local decrease of pH. Note that, while hydrogen peroxide is directly produced by the electrochemical reaction at the electrodes, hypochlorite is produced indirectly, as a byproduct of the electrochemical production of molecular chloride.

The reactions that can take place at a cathode (the negative electrode) placed in blood are the reduction of water to form molecular hydrogen:

2H₂O+2e_{M}→H₂+2(OH)⁻ [1]

And the reduction of oxygen to form either hydrogen peroxide or water. In all cases (OH)⁻ is formed as a side product, which may cause a local increase of pH.

Useful Biological Activity

Of the products listed above Cl₂ (and its hydrolysis product HClO) and H₂O₂ may have a strong biological effect on surrounding cells and tissue, since both are strong oxidizing agents which are traditionally used as disinfectants. Both these chemicals are also locally produced naturally in the body by neutrophils in order to combat infection. Both chemicals can be formed simultaneously when current is passes between two electrodes placed in blood, since Cl₂ is formed at the anode, while H₂O₂ is formed at the cathode.

In vivo Electrochemical Production of Hypochlorite

Of the four anions listed in Table 1 above, only the chlorine ion is dischargeable at the anode, leading to the production of molecular chloride, following the reaction:

2C⁻→Cl₂+2e_{M} [2]

Molecular chloride, undergoes further reactions in the presence of water, following the scheme give below:

Cl₂+H₂O→HClO+H⁺+Cl⁻ [3]

Adding Eq. [2] and Eq. [3] reveals that HClO and H⁺ are formed:

Cl⁻+H₂O→HClO+H⁺+2e_{M} [4]

An additional reaction that can take place at the anode is oxygen evolution, represented by:

2H₂O→O₂+4H⁺+4e_{M} [5]

Adding up equations 1-5, it is noted that the products are:

| | |
|---|---|
| At the cathode: | molecular hydrogen and (OH)⁻ |
| At the anode: | hypochlorous acid (HClO), H⁺ and molecular |
| | oxygen. |

The normal pH of blood is 7.4. It is noted that the electrochemical reactions taking place at the anode tend to increase the acidity (lower the pH value) of the blood, while at the cathode there is a tendency to increase the pH value of the blood. However, blood and other body fluids have inherent pH buffering properties (i.e., buffering capacity) to maintain the pH at an essentially constant level, as long as the pH perturbation is not too drastic.

The Effect of HClO on Tissue

Hypochlorous acid is a strong oxidizing agent and a well-known disinfectant. It is widely used for treatment of drinking water and as a bactericide. U.S. Pat. No. 3,725,266 issued in 1973 to Haviland teaches a method of disinfecting water by passing the water between two electrodes and applying a low-voltage signal to the electrodes. In a subsequent publication (Stoner et al., in Bioelectrochemistry and Bioengineering, 9, (1982), 229-243) it was shown that the lethal species evolved was HClO, produced at the anode. Since according to the method by Scoville the electrodes' functionality is periodically reversed (i.e., the electrode which functions as the anode and the electrode which functions as the cathode periodically reciprocate their functionality), the excess HClO formed in one half cycle is destroyed in the next half cycle. In this way a relatively high concentration of the oxidant is formed for short periods of time at the vicinity of each of the electrodes, while its bulk concentration is maintained at a low level.

In their paper Stoner et al. (ibid.) have demonstrated that the biological activity of undissociated HClO far exceeds that of its anion.

The dissociation constant of HClO or, in other words, the equilibrium constant of the reaction HClO→H⁺+ClO⁻[6] is 3.7×10⁻⁸.

Thus, the pKₐ of the reaction (i.e., −logkₐ) equals 7.4. It is well known that when the pH of a solution equals the pKₐ of an acid dissolved therein, at all times, half of the acid molecules are dissociated. Since the blood pH value equals 7.4 half of the HClO is dissociated to H⁺ and ClO⁻.

The Concentration of HClO at the Surface, as a Function of Applied Current

The concentration of the product at the electrode surface as a function of applied current density is determined by the mass balance, as dictated by the principle of mass conservation. Thus, the flux of reactant arriving at the surface (per area unit, per time unit) equals to the flux of product leaving the surface. At a current density of i (Amp/cm²) the flux of Cl⁻ ions reaching the surface is given by:

$$\frac{i}{n F} = - {{D}_{Cl -} ( \frac{\partial {C}_{{Cl}^{-}}}{\partial x} )}_{x = 0} \qquad [ 7 ]$$

where D_{I} and C_{I} represent the diffusion coefficient and concentration of the I-th species, respectively. F is the Faraday constant and n is the number of equivalents per mole.

The flux of HClO leaving the surface is the same, but with opposite sign, thus:

D_{Cl}_{⁻}(∂C_{Cl}_{⁻}/∂x)ₓ₌₀+D_{HClO}(∂C_{HClO}/∂x)ₓ₌₀=0 [8]

The flux can also be written as:

$$\frac{i}{n F} = - {{D}_{{Cl}^{-}} ( \frac{\partial {C}_{{Cl}^{-}}}{\partial x} )}_{x = 0} = \frac{{D}_{HClO} C ( x = 0 )}{δ} \qquad [ 9 ]$$

where δ is the Nernst diffusion layer thickness, and C_{I} (x=0) is the concentration of product I at the surface.

A numerical estimation yields the following results:

Assume δ=2.5×10⁻³ cm; and

D_{HClO≈D}_{Cl}⁻≈1×10⁻⁵ cm²/s

It follows from Eq. [9] above that the concentration of HClO at the electrode surface is:

$${C}_{HClO} ( x = 0 ) = \frac{i δ}{{FD}_{HClO}} \approx 2.5 \times {10}^{- 3} i \qquad [ 10 ]$$

where C_{HClO} is given in units of eq/cm and the current in units of Amp/cm². Transforming this to concentration units of ppm (parts per million) and current density of μA/cm², one obtains:

$${C}_{HClO} ( x = 0 ) = \frac{2.5 \times {10}^{- 3} \times {10}^{- 6} \times {10}^{3} \times 52.5}{1 \times {10}^{3}} i = 0.13 i [ ppm / ( \frac{µ A}{{cm}^{2}} ) ] \qquad [ 11 ]$$

The concentrations of HClO typically used for disinfection depend on the liquid being treated. One ppm HClO is usually sufficient to disinfect drinking water. A somewhat higher concentration, a few ppm is required for disinfecting public swimming pools, while when disinfecting heavily polluted streams of effluent (e.g., raw sewage), a far higher concentration is needed. In the present context of in vivo use, for the local killing of cells, such as in cases of confined proliferative disorders and diseases, including, for example, stenosis, restenosis, in-stent stenosis, solid tumors, etc., a local HClO concentration of 0.1-1.0 ppm is expected to be sufficient, corresponding to a current density of 0.8-8 μA/cm².

According to the above, the concentration of HClO decays almost linearly along a distance normal to the electrode surface, approaching zero at a distance of δ=2.5×10⁻³ cm.

Application of an “on-off-cycle” in which the “off” period is at least three times in length the “on” period can limit the volume in which HClO exists to any desired distance from the electrode surface. This distance is determined by the following equation:

δ=(πD_{HClO})^{½} [12]

For example, “on” times of 0.1 seconds and 1.0 seconds yield values of δ=1.8×10⁻³ cm and 5.6×10⁻³ cm, respectively. This is an important tool, since control of the “on” time can limit the existence of HClO to a region in a very close proximity to the electrode surface, where, for some applications, it is most needed. At the same time it prevents its spreading into the bulk of the blood, if so desired.

Differences Between Body Environment and the Simplified Aqueous Solution

The diffusion coefficients value used in the above calculations is characteristic of simple aqueous solutions at 25° C. At body temperature (37° C.) this value can be as much as 30% higher. The viscosity of blood plasma is slightly higher than that of dilute aqueous solutions (0.014-0.016 poise versus 0.010 poise, respectively). This difference should not significantly influence the calculations developed herein for simple aqueous solutions.

The Effect of pH

It is noted above that protons are released at the anode, as a side reaction of the oxidation of Cl⁻. The change of pH is not expected to be significant at the low levels of current density applied for several reasons as follows. First, the blood is naturally buffered, resisting a change of pH. Second, the diffusion coefficient of H⁺ is several times higher that of HClO (about 7 times in water). Thus, the concentration of H⁺ produced at the surface (C_{H}_{⁺}(x =o)) is expected to be several times smaller than that of HClO.

A decrease in pH near an injured tissue has been observed under certain circumstances. If this is indeed the case, it will further suppress HClO dissociation and will increase its local concentration and effectiveness.

Choice of Materials and Corrosion

The conductive materials commonly used to fabricate stents and other implants include stainless steel, tantalum, nickel/titanium alloy, iridium/platinum alloys and some stents are even coated with a thin gold layer. All these materials are stable against corrosion in blood under ordinary conditions at open circuit or when a steady anodic potential is applied. At the current levels (several μA/cm²) and electrical potential described herein it is unlikely that any significant corrosion should occur to the stent.

Some corrosion of stainless steel might occur due to the fairly high concentration of Cl⁻ ions in the blood and other body fluids that may cause a breakdown of the passive layer, when the potential is cycled in the cathodic (negative) direction. For example, in the case of gold, corrosion may be associated with formation of soluble (AuCl₄)⁻ ions.

This problem can be avoided by applying anodic pulses instead of a cyclic waveform. Thus, potential (or current) pulses are applied in the positive direction (forming HClO) followed by periods during which the electrode is left at open circuit, thus, preventing or minimizing destruction of the anodic protective film, while retaining the advantages of applying the voltage or current in pulses as discussed hereinabove.

While the pH at the anode tends to decrease, that at the cathode may increase during application of the pulse. Although this change of pH is expected to be minor, it might, under certain conditions, cause precipitation of Mg(OH)₂ at the cathode. Application of a pulsed current waveform with a short duty cycle (i.e., longer “off” periods over “on” periods) alleviates this problem. During the “off” period the pH at the cathode restores to its bulk value, and no precipitate is thus formed.

In order to prevent masking of the implant from the blood via cell growth, which may limit the access of chlorine ions to the implant, the implant is formed with groves or pitting over its surface, having a typical size smaller than the size of a cell, so as to prevent the formation of a dense cell population grown over the surface of the implant, leaving space between the cells, so as to allow the small chlorine ions to reach the implant electrode.

The Biochemistry of Chlorine

The hypochlorite molecule is a strong oxidizing agent that is toxic to cells and is used as a disinfecting chemical. U.S. Pat. No. 5,951,458 to Hastings et al. teaches the use of a strong oxidizing agent locally delivered by a catheter, for the prevention of restenosis. According to this embodiment of the present invention the oxidizing agent is locally produced by electrochemical conversion of chlorine ions into chlorine molecules that at the physiological pH hydrolyze to form hypochlorite. The released hypochlorite reacts with cells at the implant's vicinity or further downstream and prevents cell proliferation. The degree of damage to the cells and the depth of such damage depend on the local concentration of the hypochlorite. From studies on the disinfecting efficiency of hypochlorite a level of 40 ppm is required to kill all viruses and bacteria. Therefore, the local concentration of the hypochlorite should be between 0.1 ppm and 40 ppm in order to control cell growth, corresponding to a current density of 0.8-320 μA/cm².

### Example 2

In vivo Electrochemical Production of Hydrogen Peroxide

The stability of H₂O₂ in aqueous solutions containing molecular oxygen is determined by the following electrochemical reactions

H₂O₂+2H⁺+2e_{M}→2H₂O [13]

O₂+2H⁺+2e_{M}→H₂O₂ [14]

The two other reactions that can take place and are relevant in the present context are the anodic and cathodic decomposition of water, i.e., oxygen evolution at the anode and hydrogen evolution at the cathode:

O₂+4H⁺+4e_{M}→2H₂O [15]

2H₂O+4e_{M}→4(OH)⁻ [16]

The corresponding standard reduction potentials at pH=0 and at the body pH of 7.4 are:

| | | E⁰ (volt SHE) | E⁰ (volt SHE) |
|---|---|---|---|
| Equation | Reaction | at pH = 0 | at pH = 7.4 |
| 13 | Reduction of H₂O₂ to H₂O | 1.776 | 1.339 |
| 14 | Reduction of O₂ to H₂O₂ | 0.682 | 0.245 |
| 15 | Reduction of O₂ to H₂O | 1.229 | 0.792 |
| 16 | Reduction of H₂O to H₂ | 0.000 | −0.437 |

It follows from these data that hydrogen peroxide is not stable thermodynamically in water. To further demonstrate this, one may add reaction 13 with the reverse of reaction 14:

$$\begin{matrix}{H}_{2} {O}_{2} + 2 {H}^{+} + 2 {e}_{M} \to 2 {H}_{2} O {E}^{0} = 1.339 V & [ 13 ] \\ {H}_{2} {O}_{2} \to {O}_{2} + 2 {H}^{+} + 2 {e}_{M} {E}^{0} = - 0.245 V & [ - 14 ]\end{matrix} \begin{matrix}2 {H}_{2} {O}_{2} \to {O}_{2} + 2 {H}_{2} O ; Δ {E}^{0} = 1.094 V & [ 17 ]\end{matrix}$$

The self-decomposition reaction of hydrogen peroxide (Eq. 17) is favored thermodynamically since:

ΔG⁰=−nFΔE⁰=−211kJ/mole [18]

The relative stability of this compound in water is primarily due to the slow kinetics of its decomposition. This is not surprising, considering that during the reaction described in Eq. 17 two H—O bonds are broken in one molecule and an O—O bond is broken in another. It also follows from Eq. 17 that the rate of self-decomposition, which is a bi-homomolecular reaction, will decrease with dilution, as is well known experimentally.

From a thermodynamics point of view, is should not be possible to make hydrogen peroxide in aqueous solution. At the positive electrode water is oxidized to hydrogen peroxide at 1.339 V (at pH=7.4), while hydrogen peroxide is oxidized to molecular oxygen at a much lower potential of 0.245 V. In other words, at the potential at which it is formed from water, H₂O₂ is highly unstable with respect to its further oxidation to O₂.

At the negative electrode, oxygen can be reduced to hydrogen peroxide at a potential of 0.245 V, where it is highly unstable towards further reduction to water, which can occur already at a potential of 1.339 V. This is a direct consequence of the thermodynamic instability of H₂O₂.

The kinetics of the different reactions plays, however, a decisive role. In practice O₂ is reduced in two stages. A two-electron reduction step to H₂O₂ (c.f. Eq. 14) followed by another two-electron reduction step of the peroxide to water (c.f. Eq. 13). The slow kinetics of the second step is not surprising. In Eq. 14 two protons are attached to an oxygen molecule following charge transfer, but no bonds are broken. In Eq. 13 the O—O bond must be broken in addition. Indeed, one of the challenges facing the development of practical fuel cells is to develop efficient (and inexpensive) catalyst-that can promote the reduction of oxygen all the way to water and prevent its termination at the peroxide stage.

Hydrogen evolution can be a relatively fast reaction, comparable or even faster than the reduction of O₂ to H₂O₂. However, its reversible potential is 0.682 V more negative, therefore oxygen reduction to peroxide is found to occur first. The second reduction wave of oxygen, associated with the reduction of H₂O₂ that is formed as an intermediate, is at such a high overpotential in the region of hydrogen evolution that it can occur before, together with, or after the onset of hydrogen evolution.

In summary, the sequence of reactions occurring at the cathode in an aqueous solution containing oxygen is:

O₂→H₂O₂→H₂O→H₂ [19]

If the current density applied is small and the concentration of oxygen in the solution is high enough, so that its concentration at the surface is not significantly depleted, the first step, i.e., the production of H₂O₂ will probably be the only process taking place at the negative electrode (the cathode).

The concentration of free oxygen in the blood is about 0.3 cc/100 cc. This yields 3/22.4×10³ mole/liter=0.13 mM. This should be compared to a value of about 0.25 mM oxygen in water at equilibrium with air. The corresponding limiting current can be obtained from the following equation:

$${i}_{L} = \frac{nFDC}{δ} = \frac{2 \times {10}^{5} \times {10}^{- 5} \times 1.3 \times {10}^{- 7}}{2.5 \times {10}^{- 3}} \times {10}^{6} \approx 1 \times {10}^{2} µA / {cm}^{2} \qquad [ 20 ]$$

As long as the applied current density is below this value, the surface concentration depends only on the current density applied.

### Example 3

Electrochemical Reactions in the Blood and Other Body Fluids—Reactions Involving Species That do not Naturally Exist in Blood

Another possibility while implementing the present invention is to administer an electrochemically reactive reactant to the blood. Nitric oxide (NO) can be produced by reduction of NO₂⁻ or NO₃⁻.

Following are the relevant reactions and their reversible potential in the body fluid, at pH=7.4:

NO₂⁻+2H⁺+1e_{M}→NO+H₂O E=0.329 V vs. NHE [21]

NO₃⁻+4H⁺+3e_{M}→NO+2H₂O E=0.350 V vs. NHE [22]

It is clear from the above that, from a thermodynamic point of view, both reactions can occur at the cathode at readily available potentials, provided that a finite concentration of one of the reactants (the nitrite or the nitrate ion) exists in solution. Indeed, the potential for production of NO from these anions is relatively positive, so that this reaction may take preference over the reduction of oxygen to hydrogen peroxide and certainly over the evolution of molecular hydrogen. As is shown below, the concentration of reactant needed to produce the desired molecule at concentrations of the order of 1-10 ppm are quite low.

The natural concentration of nitrate (NO₃⁻) in blood is 37.7 μmole/l and that of nitrite is 262±34 nmole/l. The daily dietary intake of nitrate is about 95 mg and additional 13.5 mg it contribute by the drinking of water (in Great Britain, Knight T M, Forman D, Al-Dabbagh S A, Doll R. Food Chem. Toxicol. 1987 25(4) 277-285 Estimation of dietary intake of nitrate and nitride in Great Britain).

Consider the reaction shown in Eq. 22 above. The expression for the flux of materials to and from the surface is as follows: the flux of NO₃⁻ reaching the surface and NO leaving the surface are the same, but with opposite signs, thus:

D_{NO}_{₃}_{⁻}(∂C_{NO}_{₃}_{⁻}/∂x)ₓ₌₀+D_{NO}(∂C_{NO}/∂x)ₓ₌₀=0 [23]

The flux can also be written as:

$$\frac{i}{n F} = - {{D}_{{NO}_{3}^{-}} ( \frac{\partial {C}_{{NO}_{3}^{-}}}{\partial x} )}_{x = 0} = \frac{{D}_{NO} C ( x = 0 )}{δ} \qquad [ 24 ]$$

where δ is the Nernst diffusion layer thickness.

A numerical estimation yields the following results:

Assume δ=2.5×10⁻³ cm;

D_{NO}_{₃}_{⁻}=D_{NO} =1×10⁻⁵ cm²s⁻¹

It follows from Eq. 24 above that the concentration of NO at the surface is:

$${C}_{NO} ( x = 0 ) = \frac{i δ}{{FD}_{NO}} \approx 2.5 \times {10}^{- 3} i \qquad [ 25 ]$$

where C is in units of eq/cm³and i is in units of Amp/cm². Converting to μ A/cm² and ppm units, one finds:

$${C}_{NO} ( x = o ) = \frac{2.5 \times {10}^{- 3} \times {10}^{- 6} \times {10}^{3} \times 30}{1 \times {10}^{3}} i = 0.075 i \frac{ppm}{( µA \times {cm}^{- 2} )} \qquad [ 26 ]$$

It is important note that the above equation holds only as long as the surface concentration calculated from it does not exceed the bulk concentration of the reactant. In other words, the maximum current density for which this equation can be applied should not exceed the limiting current density for the reduction of the reactant on the surface. The latter is given by:

i_{L}(NO₃⁻)=FD_{NO}_{₃}_{⁻}C_{bulk}/δ [27]

Assuming a value of i_{L}=10 μA/cm² this yields C_{bulk}=25 μmole/l or 1.5 ppm. This is the concentration of nitrate needed to sustain a current density of 10 μA/cm², yielding a surface concentration of 0.75 ppm of NO.

### Example 4

Control via an Acoustic Switch

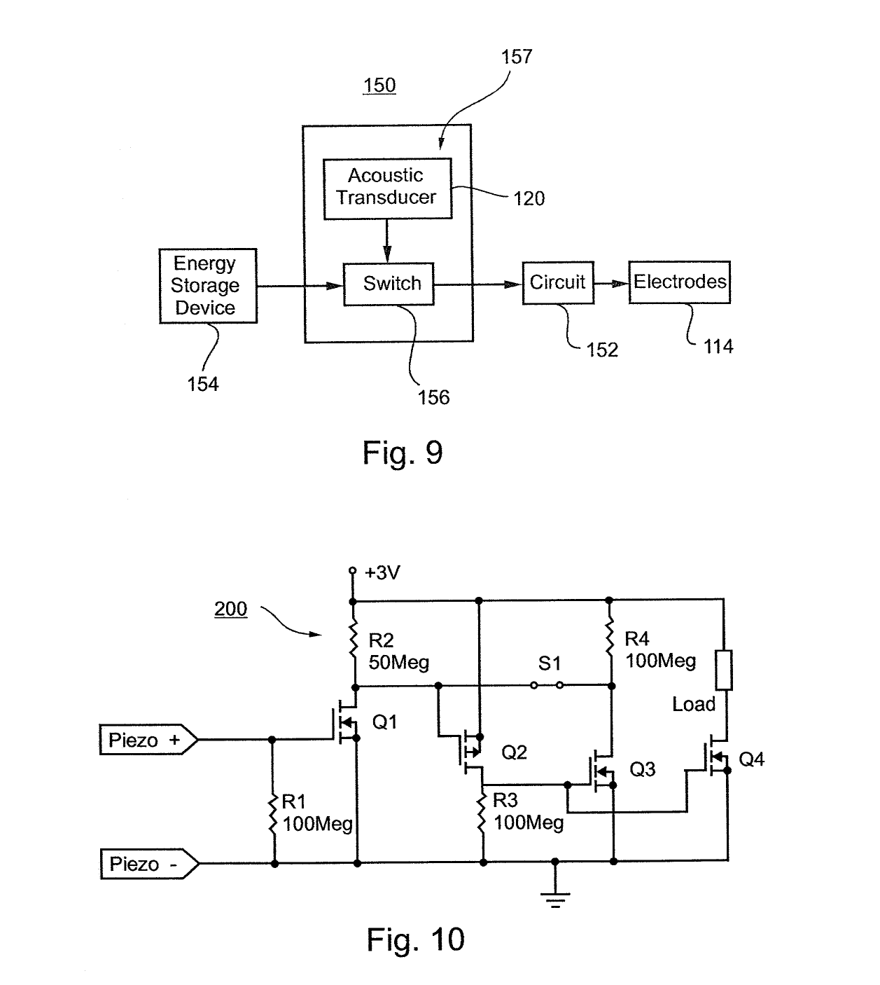

FIG. 9 shows an acoustic switch 150 which can be used in various embodiments of the invention to control power supply to the electrodes of an implant according to the invention. Acoustic switch 150 includes an electrical circuit 152 configured for performing one or more functions or commands when activated.

Acoustic switch 150 further includes an energy storage device 154 (power source) and an acoustic transducer 120 coupled to electrical circuit 152 and energy storage device 154.

In addition, acoustic switch 150 also includes a switch 156, as is further described below, although alternatively other switches, such as a miniature electromechanical switch and the like (not shown) may be provided.

Energy storage device 154 may be any of a variety of known devices, such as an energy exchanger, a battery and/or a capacitor (not shown). Preferably, energy storage device 154 is capable of storing-electrical energy substantially indefinitely. In addition, energy storage device 154 may be capable of being charged from an external source, e.g., inductively, as will be appreciated by those skilled in the art. In a preferred embodiment, energy storage device 154 includes both a capacitor and a primary, non-rechargeable battery. Alternatively, energy storage device 154 may include a secondary, rechargeable battery and/or capacitor that may be energized before activation or use of acoustic switch 150.

Acoustic switch 150 operates in one of two modes, a “sleep” or “passive” mode when not in use, and an “active” mode, when commanding electrical energy delivery from energy storage device 154 to electrical circuit 152 in order to create a potential in electrodes 114.

When in the sleep mode, there is substantially no energy consumption from energy storage device 154, and consequently, acoustic switch 150 may remain in the sleep mode virtually indefinitely, i.e., until activated. Thus, acoustic switch 150 may be more energy efficient and, therefore, may require a smaller capacity energy storage device 154 than power switching devices that continuously draw at least a small amount of current in their “passive” mode.

To activate the acoustic switch, one or more external acoustic energy waves or signals 157 are transmitted until a signal is received by acoustic transducer 150. Upon excitation by acoustic wave(s) 157, acoustic transducer 120 produces an electrical output that is used to close, open, or otherwise activate switch 156. Preferably, in order to achieve reliable switching, acoustic transducer 120 is configured to generate a voltage of at least several tenths of a volt upon excitation that may be used as an activation signal to close switch 156.

As a safety measure against false positives (either erroneous activations or deactivations), switch 156 may be configured to close only upon receipt of an initiation signal followed by a confirmation signal. For example, an activation signal that includes a first pulse followed by a second pulse separated by a predetermined delay may be employed.

In addition to an activation signal, acoustic transducer 120 may be configured for generating a termination signal in response to a second acoustic excitation (which may be of different wavelength or duration than the activation signal) in order to return acoustic switch 150 to its sleep mode.

For example, once activated, switch 156 may remain closed indefinitely, e.g., until energy storage device 154 is depleted or until a termination signal is received by acoustic transducer 120. Alternatively, acoustic switch 150 may include a timer (not shown), such that switch 156 remains closed only for a predetermined time, whereupon it may automatically open, returning acoustic switch 150 to its sleep mode.

Acoustic switch may also include a microprocessor unit which serves to interpret the electrical signal provided from acoustic transducer 120 (e.g., frequency thereof) into a signal for switching switch 156.

As shown in FIG. 10 switch circuitry 200 includes a piezoelectric transducer, or other acoustic transducer such the acoustic transducer described hereinabove (not shown, but connectable at locations piezo + and piezo −), a plurality of MOSFET transistors (Q1-Q4) and resistors (R1-R4), and switch S1.

In the switch's “sleep” mode, all of the MOSFET transistors (Q1-Q4) are in an off state. To maintain the off state, the gates of the transistors are biased by pull-up and pull-down resistors. The gates of N-channel transistors (Q1, Q3 & Q4) are biased to ground and the gate of P-channel transistor Q2 is biased to +3V. During this quiescent stage, switch S1 is closed and no current flows through the circuit.

Therefore, although an energy storage device (not shown, but coupled between the hot post, labeled with an exemplary voltage of +3V, and ground) is connected to the switch circuitry 200, no current is being drawn therefrom since all of the transistors are quiescent.

When the piezoelectric transducer detects an external acoustic signal, e.g., having a particular frequency such as the transducer's resonant frequency, the voltage on the transistor Q1 will exceed the transistor threshold voltage of about one half of a volt. Transistor Q1 is thereby switched on and current flows through transistor QI and pull-up resistor R2. As a result of the current flow through transistor Q1, the voltage on the drain of transistor Q1 and the gate of transistor Q2 drops from +3V substantially to zero (ground). This drop in voltage switches on the P-channel transistor Q2, which begins to conduct through transistor Q2 and pull-down resistor R3.

As a result of the current flowing through transistor Q2, the voltage on the drain of transistor Q2 and the gates of transistors Q3 and Q4 increases from substantially zero to +3V. The increase in voltage switches on transistors Q3 and Q4. As a result, transistor Q3 begins to conduct through resistor R4 and main switching transistor Q4 begins to conduct through the “load”, thereby switching on the electrical circuit.

As a result of the current flowing through transistor Q3, the gate of transistor Q2 is connected to ground through transistor Q3, irrespective of whether or not transistor Q1 is conducting. At this stage, the transistors (Q2, Q3 & Q4) are latched to the conducting state, even if the piezoelectric voltage on transistor Ql is subsequently reduced to zero and transistor Q1 ceases to conduct Thus, main switching transistor Q4 will remain on until switch S1 is opened.

In order to deactivate or open switch circuitry 200, switch S1 must be opened, for example, while there is no acoustic excitation of the piezoelectric transducer. If this occurs, the gate of transistor Q2 increases to +3V due to pull-up resistor R2. Transistor Q2 then switches off, thereby, in turn, switching off transistors Q3 and Q4. At this stage, switch circuitry 200 returns to its sleep mode, even if switch S1 is again closed. Switch circuitry 200 will only return to its active mode upon receiving a new acoustic activation signal from the piezoelectric transducer.

It should be apparent to one of ordinary skill in the art that the above-mentioned electrical circuit is not the only possible implementation of a switch for use with the present invention. For example, the switching operation my be performed using a CMOS circuit, which may draw less current when switched on, or by an electromechanical switch, and the like.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of producing a therapeutic agent in a body, the method comprising implanting a plurality of electrodes in the body and creating an electrical potential between pairs of said electrodes, said electrical potential being for electrochemically converting at least one substance present in the body, directly or indirectly, into the therapeutic agent.

2. The method of claim 1, wherein said at least one substance is a normal blood constituent.

3. The method of claim 2, wherein said normal blood constituent is selected from the group consisting of chlorine ions, water, hydrogen ions, hydroxide ions, molecular oxygen, nitrite and nitrate ions and L-arginine.

4. The method of claim 1, wherein said at least one substance is added to or augmented in the body.

5. The method of claim 4, wherein said at least one substance is added to or augmented in the body through a diet.

6. The method of claim 4, wherein said at least one substance is added to or augmented in the body through a medical administration.

7. The method of claim 4, wherein said at least one substance is nitrite and/or nitrate ions.

8. The method of claim 1, wherein said therapeutic agent is an oxidizing agent.

9. The method of claim 8, wherein said oxidizing agent is selected from the group consisting of molecular chloride, perchloric acid, superoxide, ozone, molecular oxygen, singlet oxygen, hydroxyl radical, hypochlorite and hydrogen peroxide.

10. The method of claim 1, wherein said therapeutic agent is nitric oxide.

11. The method of claim 1, wherein creating said electrical potential between said electrodes is effected by a battery in electrical communication with said electrodes.

12. The method of claim 1, wherein creating said electrical potential between said electrodes is effected by a telemetric energy transfer from outside the body.

13. The method of claim 12, wherein said telemetric energy transfer is selected from the group consisting of radio frequency energy transfer, magnetic energy transfer and acoustic energy transfer.

14. A method of producing an oxidizing agent in a body, the method comprising implanting a plurality of electrodes in the body and creating an electrical potential between pairs of said electrodes, said electrical potential being for electrochemically converting at least one substance present in the body, directly or indirectly, into the oxidizing agent.

15. The method of claim 14, wherein said at least one substance is a normal blood constituent.

16. The method of claim 15, wherein said normal blood constituent is selected from the group consisting of chlorine ions, water, hydrogen ions, hydroxide ions and molecular oxygen.

17. The method of claim 14, wherein said oxidizing agent is selected from the group consisting of molecular chloride, perchloric acid, superoxide, ozone, molecular oxygen, singlet oxygen, hydroxyl radical, hypochlorite and hydrogen peroxide.

18. The method of claim 14, wherein creating said electrical potential between said electrodes is effected by a battery in electrical communication with said electrodes.

19. The method of claim 14, wherein creating said electrical potential between said electrodes is effected by a telemetric energy transfer from outside the body.

20. The method of claim 19, wherein said telemetric energy transfer is selected from the group consisting of radio frequency energy transfer, magnetic energy transfer and acoustic energy transfer.

21. A method of preventing or reducing cell proliferation in a region of a body, the method comprising producing an oxidizing agent in the region of the body by implanting a plurality of electrodes in the body and creating an electrical potential between pairs of said electrodes, said electrical potential being for electrochemically converting at least one substance present in the body, directly or indirectly, into the oxidizing agent in an amount sufficient for reducing cell proliferation in the region of the body.

22. The method of claim 21, wherein said at least one substance is a normal blood constituent.

23. The method of claim 22, wherein said normal blood constituent is selected from the group consisting of chlorine ions, water, hydrogen ions, hydroxide ions and molecular oxygen.

24. The method of claim 21, wherein said oxidizing agent is selected from the group consisting of molecular chloride, perchloric acid, superoxide, ozone, molecular oxygen, singlet oxygen, hydroxyl radical, hypochlorite and hydrogen peroxide.

25. The method of claim 21, wherein creating said electrical potential between said electrodes is effected by a battery in electrical communication with said electrodes.

26. The method of claim 21, wherein creating said electrical potential between said electrodes is effected by a telemetric energy transfer from outside the body.

27. The method of claim 26, wherein said telemetric energy transfer is selected from the group consisting of radio frequency energy transfer, magnetic energy transfer and acoustic energy transfer.

28. A method of producing nitric oxide in a body, the method comprising implanting a plurality of electrodes in the body and creating an electrical potential between pairs of said electrodes, said electrical potential being for electrochemically converting at least one substance present in the body, directly or indirectly, into nitric oxide.

29. The method of claim 28, wherein said at least one substance is a normal blood constituent.

30. The method of claim 29, wherein said normal blood constituent is selected from the group consisting of nitrite and nitrate ions and L-arginine.

31. The method of claim 28, wherein said at least one substance is added to or augmented in the body.

32. The method of claim 31, wherein said at least one substance is added to or augmented in the body through a diet.

33. The method of claim 31, wherein said at least one substance is added to or augmented in the body through a medical administration.

34. The method of claim 28, wherein creating said electrical potential between said electrodes is effected by a battery in electrical communication with said electrodes.

35. The method of claim 28, wherein creating said electrical potential between said electrodes is effected by a telemetric energy transfer from outside the body.

36. The method of claim 28, wherein said telemetric energy transfer is selected from the group consisting of radio frequency energy transfer, magnetic energy transfer and acoustic energy transfer.

37. A method of vasodilating a tissue or organ in a body, the method comprising implanting a plurality of electrodes in the body and creating an electrical potential between pairs of said electrodes, said electrical potential being for electrochemically converting at least one substance present in the body, directly or indirectly, into a vasodilating agent nitric oxide.

38. The method of claim 37, wherein said at least one substance is a normal blood constituent.

39. The method of claim 38, wherein said normal blood constituent is selected from the group consisting of nitrite and nitrate ions and L-arginine.

40. The method of claim 37, wherein said at least one substance is added to or augmented in the body.

41. The method of claim 40, wherein said at least one substance is added to or augmented in the body through a diet.

42. The method of claim 40, wherein said at least one substance is added to or augmented in the body through a medical administration.

43. The method of claim 37, wherein creating said electrical potential between said electrodes is effected by a battery in electrical communication with said electrodes.

44. The method of claim 37, wherein creating said electrical potential between said electrodes is effected by a telemetric energy transfer from outside the body.

45. The method of claim 37, wherein said telemetric energy transfer is selected from the group consisting of radio frequency energy transfer, magnetic energy transfer and acoustic energy transfer.

46. A medical implant for producing a therapeutic agent in a body, the medical implant comprising a pair of electrodes and a mechanism for creating an electrical potential between pairs of said electrodes, said electrical potential being for electrochemically converting at least one substance present in the body, directly or indirectly, into the therapeutic agent.

47. The medical implant of claim 46, wherein said at least one substance is a normal blood constituent.

48. The medical implant of claim 47, wherein said normal blood constituent is selected from the group consisting of chlorine ions, water, hydrogen ions, hydroxide ions, molecular oxygen, nitrite and nitrate ions and L-arginine.

49. The medical implant of claim 46, wherein said at least one substance is added to or augmented in the body.

50. The medical implant of claim 49, wherein said at least one substance is added to or augmented in the body through a diet.

51. The medical implant of claim 49, wherein said at least one substance is added to or augmented in the body through a medical administration.

52. The medical implant of claim 49, wherein said at least one substance is nitrite and/or nitrate ions.

53. The medical implant of claim 46, wherein said therapeutic agent is an oxidizing agent.

54. The medical implant of claim 53, wherein said oxidizing agent is selected from the group consisting of molecular chloride, perchloric acid, superoxide, ozone, molecular oxygen, singlet oxygen, hydroxyl radical, hypochlorite and hydrogen peroxide.

55. The medical implant of claim 46, wherein said therapeutic agent is nitric oxide.

56. The medical implant of claim 46, wherein said mechanism for creating said electrical potential between said electrodes includes a battery in electrical communication with said electrodes.

57. The medical implant of claim 46, wherein said mechanism for creating said electrical potential between said electrodes includes an energy transducer for transducing telemetric energy received from outside the body into electric energy.

58. The medical implant of claim 57, wherein said telemetric energy is selected from the group consisting of radio frequency energy, magnetic energy and acoustic energy.

59. A medical implant for producing an oxidizing agent in a body, the medical implant comprising a pair of electrodes and a mechanism for creating an electrical potential between pairs of said electrodes, said electrical potential being for electrochemically converting at least one substance present in the body, directly or indirectly, into the oxidizing agent.

60. The medical implant of claim 59, wherein said at least one substance is a normal blood constituent.

61. The medical implant. of claim 60, wherein said normal blood constituent is selected from the group consisting of chlorine ions, water, hydrogen ions, hydroxide ions and molecular oxygen.

62. The medical implant of claim 59, wherein said oxidizing agent is selected from the group consisting of molecular chloride, perchloric acid, superoxide, ozone, molecular oxygen, singlet oxygen, hydroxyl radical, hypochlorite and hydrogen peroxide.

63. The medical implant of claim 59, wherein said mechanism for creating said electrical potential between said electrodes includes a battery in electrical communication with said electrodes.

64. The medical implant of claim 59, wherein said mechanism for creating said electrical potential between said electrodes includes an energy transducer for transducing telemetric energy received from outside the body into electric energy.

65. The medical implant of claim 64, wherein said telemetric energy is selected from the group consisting of radio frequency energy, magnetic energy and acoustic energy.

66. A medical implant characterized by preventing cell proliferation in its vicinity via producing an oxidizing agent in a body, the medical implant comprising a pair of electrodes and a mechanism for creating an electrical potential between pairs of said electrodes, said electrical potential being for electrochemically converting at least one substance present in the body, directly or indirectly, into the oxidizing agent.

67. The medical implant of claim 66, wherein said at least one substance is a normal blood constituent.

68. The medical implant of claim 67, wherein said normal blood constituent is selected from the group consisting of chlorine ions, water, hydrogen ions, hydroxide ions and molecular oxygen.

69. The medical implant of claim 66, wherein said oxidizing agent is selected from the group consisting of molecular chloride, perchloric acid, superoxide, ozone, molecular oxygen, singlet oxygen, hydroxyl radical, hypochlorite and hydrogen peroxide.

70. The medical implant of claim 66, wherein said mechanism for creating said electrical potential between said electrodes includes a battery in electrical communication with said electrodes.

71. The medical implant.of claim 66, wherein said mechanism for creating said electrical potential between said electrodes includes an energy transducer for transducing telemetric energy received from outside the body into electric energy.

72. The medical implant of claim 71, wherein said telemetric energy is selected from the group consisting of radio frequency energy, magnetic energy and acoustic energy.

73. A medical implant for producing nitric oxide in a body, the medical implant comprising a pair of electrodes and a mechanism for creating an electrical potential between pairs of said electrodes, said electrical potential being for electrochemically converting at least one substance present in the body, directly or indirectly, into the nitric oxide.

74. The medical implant of claim 73, wherein said at least one substance is a normal blood constituent.

75. The medical implant of claim 74, wherein said normal blood constituent is selected from the group consisting of nitrite and nitrate ions and L-arginine.

76. The medical implant of claim 73, wherein said at least one substance is added to or augmented in the body.

77. The medical implant of claim 76, wherein said at least one substance is added to or augmented in the body through a diet.

78. The medical implant of claim 76, wherein said at least one substance is added to or augmented in the body through a medical administration.

79. The medical implant of claim 76, wherein said at least one substance is nitrite and/or nitrate ions.

80. The medical implant of claim 73, wherein said mechanism for creating said electrical potential between said electrodes includes a battery in electrical communication with said electrodes.

81. The medical implant of claim 73, wherein said mechanism for creating said electrical potential between said electrodes includes an energy transducer for transducing telemetric energy received from outside the body into electric energy.

82. The medical implant of claim 81, wherein said telemetric energy is selected from the group consisting of radio frequency energy, magnetic energy and acoustic energy.

83. A medical implant for vasodilating a tissue or organ in a body, the medical implant comprising a pair of electrodes and a mechanism for creating an electrical potential between pairs of said electrodes, said electrical potential being for electrochemically converting at least one substance present in the body, directly or indirectly, into a vasodilating agent nitric oxide.

84. The medical implant of claim 83, wherein said at least one substance is a normal blood constituent.

85. The medical implant of claim 84, wherein said normal blood constituent is selected from the group consisting of nitrite and nitrate ions and L-arginine.

86. The medical implant of claim 83, wherein said at least one substance is added to or augmented in the body.

87. The medical implant of claim 86, wherein said at least one substance is added to or augmented in the body through a diet.

88. The medical implant of claim 86, wherein said at least one substance is added to or augmented in the body through a medical administration.

89. The medical implant of claim 86, wherein said at least one substance is nitrite and/or nitrate ions.

90. The medical implant of claim 83, wherein said mechanism for creating said electrical potential between said electrodes includes a battery in electrical communication with said electrodes.

91. The medical implant of claim 83, wherein said mechanism for creating said electrical potential between said electrodes includes an energy transducer for transducing telemetric energy received from outside the body into electric energy.

92. The medical implant of claim 91, wherein said telemetric energy is selected from the group consisting of radio frequency energy, magnetic energy and acoustic energy.

93. A stent comprising a stent body, a pair of electrodes and a mechanism for creating an electrical potential between pairs of said electrodes, said electrical potential being for electrochemically converting at least one substance present in the body, directly or indirectly, into the therapeutic agent.

94. The stent of claim 93, wherein said stent body is made, at least in part, of a metal and serves as one of said electrodes.

95. The stent of claim 93, wherein said at least one substance is a normal blood constituent.

96. The stent of claim 95, wherein said normal blood constituent is selected from the group consisting of chlorine ions, water, hydrogen ions, hydroxide ions, molecular oxygen, nitrite and nitrate ions and L-arginine.

97. The stent of claim 93, wherein said at least one substance is added to or augmented in the body.

98. The stent of claim 97, wherein said at least one substance is added to or augmented in the body through a diet.

99. The stent of claim 97, wherein said at least one substance is added to or augmented in the body through a medical administration.

100. The stent of claim 97, wherein said at least one substance is nitrite and/or nitrate ions.

101. The stent of claim 93, wherein said therapeutic agent is an oxidizing agent.

102. The stent of claim 101, wherein said oxidizing agent is selected from the group consisting of molecular chloride, perchloric acid, superoxide, ozone, molecular oxygen, singlet oxygen, hydroxyl radical, hypochlorite and hydrogen peroxide.

103. The stent of claim 93, wherein said therapeutic agent is nitric oxide.

104. The stent of claim 93, wherein said mechanism for creating said electrical potential between said electrodes includes a battery in electrical communication with said electrodes.

105. The stent of claim 93, wherein said mechanism for creating said electrical potential between said electrodes includes an energy transducer for transducing telemetric energy received from outside the body into electric energy.

106. The stent of claim 105, wherein said telemetric energy is selected from the group consisting of radio frequency energy, magnetic energy and acoustic energy.

107. An artificial implantable vessel comprising a vessel body, a pair of electrodes and a mechanism for creating an electrical potential between pairs of said electrodes, said electrical potential being for electrochemically converting at least one substance present in the body, directly or indirectly, into the therapeutic agent.

108. The artificial implantable vessel of claim 1, wherein said vessel body is made, at least in part, of a metal and serves as one of said electrodes.

109. The artificial implantable vessel of claim 107, wherein said at least one substance is a normal blood constituent.

110. The artificial implantable vessel of claim 109, wherein said normal blood constituent is selected from the group consisting of chlorine ions, water, hydrogen ions, hydroxide ions, molecular oxygen, nitrite and nitrate ions and L-arginine.

111. The artificial implantable vessel of claim 107, wherein said at least one substance is added to or augmented in the body.

112. The artificial implantable vessel of claim 111, wherein said at least one substance is added to or augmented in the body through a diet.

113. The artificial implantable vessel of claim 111, wherein said at least one substance is added to or augmented in the body through a medical administration.

114. The artificial implantable vessel of claim lll, wherein said at least one substance is nitrite and/or nitrate ions.

115. The artificial implantable vessel of claim 107, wherein said therapeutic agent is an oxidizing agent.

116. The artificial implantable vessel of claim 115, wherein said oxidizing agent is selected from the group consisting of molecular chloride, perchloric acid, superoxide, ozone, molecular oxygen, singlet oxygen, hydroxyl radical, hypochlorite and hydrogen peroxide.

117. The artificial implantable vessel of claim 107, wherein said therapeutic agent is nitric oxide.

118. The artificial implantable vessel of claim 107, wherein said mechanism for creating said electrical potential between said electrodes includes a battery in electrical communication with said electrodes.

119. The artificial implantable vessel of claim 107, wherein said mechanism for creating said electrical potential between said electrodes includes an energy transducer for transducing telemetric energy received from outside the body into electric energy.

120. The artificial implantable vessel of claim 119, wherein said telemetric energy is selected from the group consisting of radio frequency energy, magnetic energy and acoustic energy.

121. A method of prevention cell proliferation in a body region, the method comprising implanting in the body region or in a blood vessel feeding said body region an implant designed and constructed for the electrochemical production of an oxidizing agent.

122. The method of claim 121, wherein said oxidizing agent is selected from the group consisting of molecular chloride, perchloric acid, superoxide, ozone, molecular oxygen, singlet oxygen, hydroxyl radical, hypochlorite and hydrogen peroxide.

123. The method of claim 121, wherein said electrochemical production of said oxidizing agent is by electrochemical conversion of at least one substance present in the body, directly or indirectly, into said oxidizing agent.

124. The method of claim 123, wherein said at least one substance is a normal blood constituent.

125. The method of claim 124, wherein said normal blood constituent is selected from the group consisting of chlorine ions, water, hydrogen ions, hydroxide ions and molecular oxygen.

126. The method of claim 121, wherein creating said electrical potential between said electrodes is effected by a battery in electrical communication with said electrodes.

127. The method of claim 121, wherein creating said electrical potential between said electrodes is effected by a telemetric energy transfer from outside the body.

128. The method of claim 127, wherein said telemetric energy transfer is selected from the group consisting of radio frequency energy transfer, magnetic energy transfer and acoustic energy transfer.

129. A method of vasodilating a body region, the method comprising implanting in the body region or in a blood vessel feeding said body region an implant designed and constructed for the electrochemical production of a vasodilating agent nitric oxide.

130. The method of claim 129, wherein said electrochemical production of said nitric oxide is by electrochemical conversion of at least one substance present in the body, directly or indirectly, into said oxidizing agent.

131. The method of claim 130, wherein said at least one substance is a normal blood constituent.

132. The method of claim 131, wherein said normal blood constituent is selected from the group consisting of nitrite and nitrate ions and L-arginine.

133. The method of claim 130, wherein said at least one substance is added to or augmented in the body.

134. The method of claim 133, wherein said at least one substance is added to or augmented in the body through a diet.

135. The method of claim 133, wherein said at least one substance is added to or augmented in the body through a medical administration.

136. The method of claim 133, wherein said at least one substance is nitrite and/or nitrate ions.

137. The method of claim 129, wherein creating said electrical potential between said electrodes is effected by a battery in electrical communication with said electrodes.

138. The method of claim 129, wherein creating said electrical potential between said electrodes is effected by a telemetric energy transfer from outside the body.

139. The method of claim 138, wherein said telemetric energy transfer is selected from the group consisting of radio frequency energy transfer, magnetic energy transfer and acoustic energy transfer.

140. The method of claim 11, wherein power supply from said battery is controlled via a logic chip communicating therewith or via telemetric energy transfer.

141. The method of claim 18, wherein power supply from said battery is controlled via a logic chip communicating therewith or via telemetric energy transfer.

142. The method of claim 25, wherein power supply from said battery is controlled via a logic chip communicating therewith or via telemetric energy transfer.

143. The method of claim 28, wherein power supply from said battery is controlled via a logic chip communicating therewith or via telemetric energy transfer.

144. The method of claim 43, wherein power supply from said battery is controlled via a logic chip communicating therewith or via telemetric energy transfer.

145. The method of claim 126, wherein power supply from said battery is controlled via a logic chip communicating therewith or via telemetric energy transfer.

146. The method of claim 137, wherein power supply from said battery is controlled via a logic chip communicating therewith or via telemetric energy transfer.

147. The artificial implant of claim 56, wherein power supply from said battery is controlled via a logic chip communicating therewith or via telemetric energy transfer.

148. The artificial implant of claim 63, wherein power supply from said battery is controlled via a logic chip communicating therewith or via telemetric energy transfer.

149. The artificial implant of claim 70, wherein power supply from said battery is controlled via a logic chip communicating therewith or via telemetric energy transfer.

150. The artificial implant of claim 80, wherein power supply from said battery is controlled via a logic chip communicating therewith or via telemetric energy transfer.

151. The artificial implant of claim 90, wherein power supply from said battery is controlled via a logic chip communicating therewith or via telemetric energy transfer.

152. The stent of claim 104, wherein power supply from said battery is controlled via a logic chip communicating therewith or via telemetric energy transfer.

153. The artificial implantable vessel of claim 118, wherein power supply from said battery is controlled via a logic chip communicating therewith or via telemetric energy transfer.
